(19) United States
(12) Patent Application Publication
(10) Pub. No.: US 2018/0280474 A1
(43) Pub. Date: Oct. 4, 2018

(54) TREATMENT OF BILE ACID DISORDERS

(71) Applicant: AMGEN INC., Thousand Oaks, CA (US)

(72) Inventors: Jing Xu, Thousand Oaks, CA (US); Shanaka Stanislaus, Thousand Oaks, CA (US); Mei-Hsiu M. Chen, Agoura Hills, CA (US); Clarence H. Hale, Camarillo, CA (US); Murielle Veniant-Ellison, Thousand Oaks, CA (US)

(21) Appl. No.: 15/765,144

(22) PCT Filed: Sep. 30, 2016
(86) PCT No.: PCT/US16/55017

§ 371 Date: Mar. 30, 2018

Related U.S. Application Data
(60) Provisional application No. 62/236,050, filed on Oct. 1, 2015.

(51) Int. Cl. A61K 38/18; A61P 1/00; A61P 1/16

(52) U.S. Cl. CPC A61K 38/1825; A61P 1/16; A61P 1/00

(57) ABSTRACT

The invention relates method of treating a patient in need thereof with a long acting agonist to the FGF21 signaling pathway. In a particular embodiment, the invention relates to the use of molecules that stimulate the FGF21 signaling pathway, such as long acting FGF21 polypeptides or agonist antibodies, to treat disorders or diseases associated with excess bile acid. The invention further relates to pharmaceutical formulations and dosing of long acting agonists of the FGF21 signaling pathway suitable for treating bile acid related disorders.

### CROSS-REFERENCE TO RELATED APPLICATIONS

This application claims the benefit of U.S. Provisional Application No. 62/236,050, filed Oct. 1, 2015, which is incorporated by reference in its entirety.

### DESCRIPTION OF THE TEXT FILE SUBMITTED ELECTRONICALLY

The contents of the text file submitted electronically herewith are incorporated herein by reference in their entirety: a computer readable format copy of the Sequence Listing (filename: A-1943-WO-PCT-SeqList093016_ST25.txt, date recorded: Sep. 20, 2016, file size 17 kilobytes).

### BACKGROUND OF THE INVENTION

### Field of the Invention

The invention relates method of treating a patient in need thereof with a long acting agonist to the FGF21 signaling pathway. In a particular embodiment, the invention relates to the use of molecules that stimulate the FGF21 signaling pathway, such as long acting FGF21 polypeptides or agonist antibodies, to treat disorders or diseases associated with excess bile acid. The invention further relates to pharmaceutical formulations and dosing of long acting agonists of the FGF21 signaling pathway suitable for treating bile acid related disorders.

### Background of the Invention

Fibroblast Growth Factor 21 (FGF21) is a secreted polypeptide that belongs to a subfamily of Fibroblast Growth Factors (FGFs) that includes FGF19, FGF21, and FGF23 (Itoh et al., (2004) Trend Genet. 20:563-69). FGF21 is an atypical FGF in that it is heparin independent and functions as a hormone in the regulation of glucose, lipid, and energy metabolism.

It is highly expressed in liver and pancreas and is the only member of the FGF family to be primarily expressed in liver. Transgenic mice overexpressing FGF21 exhibit metabolic phenotypes of slow growth rate, low plasma glucose and triglyceride levels, and an absence of age-associated type 2 diabetes, islet hyperplasia, and obesity. Pharmacological administration of recombinant FGF21 protein in rodent and primate models results in normalized levels of plasma glucose, reduced triglyceride and cholesterol levels, and improved glucose tolerance and insulin sensitivity. In addition, FGF21 reduces body weight and body fat by increasing energy expenditure, physical activity, and metabolic rate. Experimental research provides support for the pharmacological administration of FGF21 for the treatment of type 2 diabetes, obesity, dyslipidemia, and other metabolic conditions or disorders in humans.

FGF21 is a liver derived endocrine hormone that stimulates glucose uptake in adipocytes and lipid homeostasis through the activation of its receptor. Interestingly, in addition to the canonical FGF receptor, the FGF21 receptor also comprises the membrane associated β-Klotho as an essential cofactor. Activation of the FGF21 receptor leads to multiple effects on a variety of metabolic parameters.

In mammals, FGFs mediate their action via a set of four FGF receptors, FGFR1-4, that in turn are expressed in multiple spliced variants, e.g., FGFR1c. FGFR2c, FGFR3c and FGFR4. Each FGF receptor contains an intracellular tyrosine kinase domain that is activated upon ligand binding, leading to downstream signaling pathways involving MAPKs (Erk1/2), RAF1, AKT1 and STATs. (Kharitonenkov et al., (2008) BioDrugs 22:37-44). Several reports suggested that the “c”-reporter splice variants of FGFR1-3 exhibit specific affinity to β-Klotho and could act as endogenous receptor for FGF21 (Kurosu et al., (2007) J. Biol. Chem. 282:26687-26695); Ogawa et al., (2007) Proc. Natl. Acad. Sci. USA 104:7432-7437); Kharitonenkov et al., (2008) J. Cell Physiol. 215:1-7). In the liver, which abundantly expresses both β-Klotho and FGFR4, FGF21 does not induce phosphorylation of MAPK albeit the strong binding of FGF21 to the β-Klotho-FGFR4 complex. In 3T3-L1 cells and white adipose tissue, FGFR1 is by far the most abundant receptor, and it is therefore most likely that FGF21's main functional receptors in this tissue are the β-Klotho/FGFR1c complexes.

Bile acid synthesis also occurs in the liver and is necessary for fatty acid absorption after a meal, but can have destructive properties if retained in excess in the liver. The most common causes of adult chronic cholestasis are primary biliary cirrhosis (PBC) and primary sclerosing cholangitis (PSC). PBC is caused by chronic, immune-mediated destruction of the small-to-medium-sized bile ducts in the liver. PSC is characterized by the destruction of the intra- or extra-hepatic large bile ducts due to autoimmune injury, toxic biliary damage, infectious triggers and vascular insults. The prevalence of PSC and PBC is about 0.6-40 per 100,000 people and 0.2-14 per 100,000 people, respectively. Other etiologies of chronic cholestatic liver diseases in adults include drug-induced cholangitis and cholestasis, contraceptive-induced cholestasis, intrahepatic cholestasis of pregnancy, intestinal failure associated liver disease, immunoglobulin G4-associated cholangitis, sarcoidosis, lymphoma and idiopathic adulthood ductopenia, bile duct injury due to rejection of transplant liver, graft-versus-host disease, long-term parenteral nutrition, cryptogenic biliary fibrosis/cirrhosis, sepsis-associated cholestasis. Chronic cholestasis can also be induced by mechanical blockage of the bile duct from gallstone, tumor or cysts. This type of cholestasis is known as obstructive cholestasis and is distinguished from metabolic cholestasis caused by genetic and acquired metabolic defects.

There are limited options for the management of cholestatic liver diseases. Currently there is no FDA approved drug for PSC. For PBC and a limited group of other cholestatic liver diseases, ursodeoxycholic acid (UDCA) is the only FDA-approved drug. UDCA is a hydrophilic natural bile acid found as a major primary bile acid in bears and a minor secondary bile acid in human. The mechanism of action of UDCA is to replace toxic hydrophobic bile acids and to make the bile acid pool more hydrophilic. Therefore, UDCA is a displacement therapy and not a cure. In addition, not all patients respond to UDCA treatment and liver transplantation is the ultimate solution for these late-stage patients. Thus there exists a need for effective treatments to reduce bile acid in patients in need thereof.

Provided herein is the first description of a class of FGF21 pathway stimulating molecules that are shown to reduce bile acid synthesis and accumulation. Representative examples of this class of FGF21 molecules include those engineered for extended half-life and antibodies that agonize the FGF21 signaling pathway through β-Klotho.

### SUMMARY OF THE INVENTION

The present disclosure provides a method to treat disorders or diseases associated with bile acid production. More particularly, disclosed herein is the use of FGF21 pathway activating molecules to reduce bile acid levels. Even more particularly are provided molecules having a longer half-life than FGF21 that signal through the FGF21 pathway and thereby reduce bile acid.

In addition to the surprising result that activation of FGF21 signaling pathways reduced both biomarkers for bile acid production and reduced the amount of bile acids in various biological locations, the present inventors also discovered that this effect was seen primarily with binding proteins having extended half-lives relative to FGF21. Certain non-limiting examples of half-life extended molecules for practice in the methods and uses of the invention include FGF21 fused to antibody Fc domains and/or FGF21 with point mutations to protect from proteolysis and/or aggregation fused to domains to extend serum half-life and antibodies that activate or agonize the FGF21 signaling pathway utilizing β-Klotho.

Thus, in certain embodiments the invention relates to the use of extended half-life FGF21 molecules to reduce bile acid levels. In other embodiments the invention relates to the use of antibodies that activate the FGF21 signaling pathway to reduce bile acid levels. In yet other embodiments, the invention relates to reduction of biomarkers associated with bile acid production, such as CYP7A1, CYP8B1, CYP27A1, CYP7B1 and 7α-Hydroxy-4-cholesten-3-one (C4). In other embodiments, the invention relates to the use of extended half-life FGF21 molecules to reduce or repair damage to the liver caused by excess bile acid accumulation.

Exemplary indications for which reduction of bile acid levels is desired include progressive familial intrahepatic cholestasis type 2 and 3 (BSEP and MDR3 mutations respectively; these are pumps that export bile acids and phospholipid out of liver), intrahepatic cholestasis of pregnancy (ICP), drug-induced cholestasis, contraceptive-induced cholestasis, primary biliary cirrhosis (autoimmune), primary sclerosing cholangitis (autoimmune), cryptogenic biliary fibrosis/cirrhosis, total parenteral nutrition (TPN)-induced cholestasis, bile duct injury following liver transplantation, sepsis-associated cholestasis, progressive sclerosing cholangitis, idiopathic adulthood ductopenia, oriental cholangiohepatitis, and cholangiopathy associated with primary hepatolithiasis.

Agonists of the FGF21 signaling pathway include various modalities including engineered FGF21 and agonist antibodies. One of skill the art will appreciate other binding proteins with half-lives extended beyond FGF21 and capable of activating the same signaling pathway are within the scope of the invention.

An example of mature, secreted human FGF21 sequence is as follows: HPIPDSSPLLQFGGQVRQRYLYTDDAQQTEAHLEIREDGTVGGAADQS PESLLQLKALKPGVIQILGVKTSRFLCQRPDGALYGSLHFDPEACSFRELLLED GYNVYQSEAHGLPLHLPGNKSPHRDPAPRGPARFLPLPGLPPAPPEPPGILAPQ PPDVGSSDPLSMVGPSQGRSPSYAS (SEQ ID NO: 1). Accordingly, the present disclosure provides an isolated polypeptide suitable for treating bile acid related disorders comprising an amino acid sequence of SEQ ID NO: 1 having: (a) at least one amino acid substitution that is: (i) a glutamine, isoleucine, or lysine residue at position 19; (ii) a histidine, leucine, or phenylalanine residue at position 20; (iii) an isoleucine, phenylalanine, tyrosine, or valine residue at position 21; (iv) an isoleucine, phenylalanine, or valine residue at position 22; (v) an alanine or arginine residue at position 150; (vi) an alanine or valine residue at position 151; (vii) a histidine, leucine, phenylalanine, or valine residue at position 152; (viii) an alanine, asparagine, aspartic acid, cysteine, glutamic acid, glutamine, proline, or serine residue at position 170; (ix) an alanine, arginine, asparagine, aspartic acid, cysteine, glutamic acid, glutamine, glycine, histidine, lysine, serine, threonine, tryptophan, or tyrosine residue at position 171; (x) a leucine or threonine residue at position 172; or (xi) an arginine or glutamic acid residue at position 173; and (b) at least one amino acid substitution that is: (i) an arginine, glutamic acid, or lysine residue at position 26; (ii) an arginine, glutamic acid, glutamine, lysine, or threonine residue at position 45; (iii) a threonine residue at position 52; (iv) a cysteine, glutamic acid, glycine, or serine residue at position 58; (v) an alanine, arginine, glutamic acid, or lysine residue at position 60; (vi) an alanine, arginine, cysteine, or histidine residue at position 78; (vii) a cysteine or threonine residue at position 86; (viii) an alanine, arginine, glutamic acid, lysine, or serine residue at position 88; (ix) an arginine, cysteine, glutamic acid, glutamine, lysine, or threonine residue at position 98; (x) an arginine, aspartic acid, cysteine, or glutamic acid residue at position 99; (xi) a lysine or threonine residue at position 111; (xii) an arginine, asparagine, aspartic acid, glutamic acid, glutamine, histidine, or lysine residue at position 129; or (xiii) an arginine, glutamic acid, histidine, lysine, or tyrosine residue at position 134; and combinations thereof. In one embodiment the residue at position 98 is arginine and the residue at position 171 is proline, and in another embodiment the polypeptide can comprise an amino acid sequence that is at least 85 percent identical to the amino acid sequence of SEQ ID NO: 1, but wherein the at least one amino acid substitution of (a)(i)-(xi) and (b)(i)-(xiii) is not further modified.

The present disclosure additionally provides an isolated polypeptide suitable for treating bile acid related disorders comprising an amino acid sequence of SEQ ID NO: 1 having at least one amino acid substitution that is: (a) a glutamine, lysine or isoleucine residue at position 19; (b) a histidine, leucine, or phenylalanine residue at position 20; (c) an isoleucine, phenylalanine, tyrosine, or valine residue at position 21; (d) an isoleucine, phenylalanine, or valine residue at position 22; (e) an alanine or arginine residue at position 150; (f) an alanine or valine residue at position 151; (g) a histidine, leucine, phenylalanine, or valine residue at position 152; (h) an alanine, aspartic acid, cysteine, or proline residue at position 170; (i) an alanine, arginine, asparagine, aspartic acid, cysteine, glutamic acid, glutamine, glycine, histidine, lysine, serine, threonine, tryptophan, or tyrosine residue at position 171; (j) a leucine residue at position 172; or (k) an arginine or glutamic acid residue at position 173; and combinations thereof. In one embodiment the residue at position 171 is proline, and in another embodiment the polypeptide can comprise an amino acid sequence that is at least 85 percent identical to the amino acid sequence of SEQ ID NO: 1, but wherein the at least one amino acid substitution of (a)-(k) is not further modified.

The present disclosure further provides an isolated polypeptide suitable for treating bile acid related disorders comprising an amino acid sequence of SEQ ID NO: 1 having at least one amino acid substitution that is: (a) an arginine, glutamic acid, or lysine residue at position 26; (b) an arginine, glutamic acid, glutamine, lysine, or threonine residue at position 45; (c) a threonine residue at position 52; (d) a glutamic acid, glycine, or serine residue at position 58; (e) an alanine, arginine, glutamic acid, or lysine residue at position 60; (f) an alanine, arginine, or histidine residue at position 78; (g) an alanine residue at position 88; (h) an arginine, glutamic acid, glutamine, lysine, or threonine residue at position 98; (i) an arginine, aspartic acid, cysteine, or glutamic acid residue at position 99; (j) a lysine or threonine residue at position 111; (k) an arginine, asparagine, aspartic acid, glutamic acid, glutamine, histidine, or lysine residue at position 129; or (1) an arginine, glutamic acid, histidine, lysine, or tyrosine residue at position 134; and combinations thereof. In one embodiment, the residue at position 98 is arginine and in another embodiment the polypeptide can comprise an amino acid sequence that is at least 85 percent identical to the amino acid sequence of SEQ ID NO: 1, but wherein the at least one amino acid substitution of (a)-(l) is not further modified.

In various embodiments, the polypeptides suitable for treating bile acid related disorders and disclosed herein can further comprise at least one amino acid substitution that is: (a) a phenylalanine, proline, alanine, serine or glycine at position 179; (b) a glutamic acid, glycine, proline, or serine at position 180; or (c) a lysine, glycine, threonine, alanine, leucine, or proline at position 181 and can further comprise 1 to 10 amino acid residues fused to the C-terminus of the polypeptide, and can be any amino acid, for example, one or more residues selected from the group consisting of glycine, proline and combinations thereof.

In various embodiments, the polypeptides suitable for treating bile acid related disorders disclosed herein can comprise (a) an amino-terminal truncation of no more than 8 amino acid residues, wherein the polypeptide is capable of lowering blood glucose in a mammal; (b) a carboxyl-terminal truncation of no more than 12 amino acid residues, wherein the polypeptide is capable of lowering blood glucose in a mammal; or (c) an amino-terminal truncation of no more than 8 amino acid residues and a carboxyl-terminal truncation of no more than 12 amino acid residues, wherein the polypeptide is capable of lowering blood glucose in a mammal.

In some embodiments, the polypeptides suitable for treating bile acid related disorders disclosed herein can be covalently linked to one or more polymers, such as PEG. In other embodiments, the polypeptides of the present invention can be fused to a heterologous amino acid sequence, optionally via a linker, such as GGGGSGGGGGSGGGGS (SEQ ID NO: 5). Fusion polypeptides disclosed herein can also form multimers.

The present disclosure also provides pharmaceutical compositions suitable for treating bile acid related disorders comprising the polypeptides disclosed herein and a pharmaceutically acceptable formulation agent. Such pharmaceutical compositions can be used in a method for treating a metabolic disorder, and the method comprises administering to a human patient in need thereof a pharmaceutical composition of the present invention. Metabolic disorders that can be treated include diabetes and obesity.

The present disclosure additionally provides an isolated fusion protein suitable for treating bile acid related disorders that can comprise: (a) an IgG constant domain: (b) a linker sequence fused to the IgG constant domain; and (c) an FGF21 mutant fused to the linker sequence and comprising the amino acid sequence of SEQ ID NO: 1 wherein the an arginine residue has been substituted for the leucine residue at position 98 and a glycine residue has been substituted for the proline residue at position 171. In one embodiment, the linker sequence can comprise GGGGSGGGSGGGGS (SEQ ID NO: 5) and in another the IgG constant domain can comprise SEQ ID NO: 11. In another embodiment, the linker sequence comprises GGGGGSGGGSGGGGS (SEQ ID NO: 5) and the IgG constant domain comprises the amino acid sequence of SEQ ID NO: 11. In still another embodiment the N terminus of the linker is fused to the C terminus of the IgG constant domain and the N terminus of the FGF21 mutant is fused to the C terminus of the linker. The disclosed fusion proteins can form multimers.

In various embodiments of the fusion protein, the FGF21 component can comprise at least one amino acid substitution that is: (a) a phenylalanine, proline, alanine, serine or glycine at position 179; (b) a glutamic acid, glycine, proline, or serine at position 180; or (c) a lysine, glycine, threonine, alanine, leucine, or proline at position 181 and can further comprise 1 to 10 amino acid residues fused to the C-terminus of the FGF21 mutant, and the 1 to 10 amino acid residues, and can be any amino acid, for example, one or more residues selected from the group consisting of glycine, proline and combinations thereof.

In specific non-limiting embodiments, point mutations are made within the FGF21 sequence at amino acid positions L98 to R and P171 to G. In other embodiments, point mutations are made with the FGF21 portion of the sequence at amino acid positions L98 to R, P 171 to G, and A180 to E. These variants can then be fused to half-life extending moieties, such as polyethylene glycol (PEG), albumin, dextran, or an Fc region as representative examples of protein half-life extending techniques.

In still other embodiments of the fusion protein, the FGF21 component can comprise: (a) an amino-terminal truncation of no more than 8 amino acid residues, wherein the polypeptide is capable of lowering blood glucose in a mammal; (b) a carboxyl-terminal truncation of no more than 12 amino acid residues, wherein the polypeptide is capable of lowering blood glucose in a mammal; or (c) an amino-terminal truncation of no more than 8 amino acid residues and a carboxyl-terminal truncation of no more than 12 amino acid residues, wherein the polypeptide is capable of lowering blood glucose in a mammal. In another embodiment, the FGF21 component of a fusion protein can comprise an amino acid sequence that is at least 85 percent identical to the amino acid sequence of SEQ ID NO: 1, but wherein the arginine and glycine residues are not further modified.

Further binding moieties are contemplated that activate the FGF21 signaling pathway and include, for example, antibodies.

The present disclosure also provides pharmaceutical compositions suitable for treating bile acid related disorders comprising the fusion protein disclosed herein and a pharmaceutically acceptable formulation agent. Such pharmaceutical compositions can be used in a method for treating a metabolic disorder, the method comprising administering to a human patient in need thereof a pharmaceutical composition of the present invention. Metabolic disorders that can be treated include diabetes and obesity.

Also provided are isolated nucleic acid molecules encoding the polypeptides of disclosed herein, as well as vectors comprising such nucleic acid molecules and host cells comprising such nucleic acid molecules.

Specific embodiments of the present invention will become evident from the following more detailed description of certain embodiments and the claims.

### BRIEF DESCRIPTION OF THE DRAWINGS

FIGS. 1A-1E: Acute effects of FGF21 on CYP7A1 Expression. Mice were injected with a single dose of FGF21. Tissues were harvested, extracted for total RNA, and analyzed for gene expression using qRT-PCR. A: Tissues collected from ad lib fed or fasted (3 or 12 hr) DIO mice 3 hours post injection. B: Time-course evaluation of FGF21 effects on CYP7A1 expression compared to plasma rhFGF21 concentration. C-E: Male DIO, C57BL6J and ob/ob mice were treated with FGF21 at indicated doses to determine treatment effects on CYP7A1 expression. All data represent mean±SEM. N=4-5 mice per group.

FIGS. 2A-2C: Acute effects of FGF21 on CYP7A Expression and other Bile Acid Metabolism Genes. Expression analysis of genes involved hepatic bile acid synthesis (A), bile acid and sterol transport (B), and ileal bile acid re-absorption (C). Each bar represents duplicate analysis of pooled samples from n=5 mice. Additionally, CYP7A1 was analyzed from individual mice and represented as mean t SEM (n=5 per group).

FIGS. 3A-1-3A-6 and 3B-1-3B-4: Effects of chronic rhFGF21 and Analog administration on Bile Acid levels in C57BL6 mice. Tissues were harvested at the termination following a 3 hour fasting and post-injection for bile acid analysis. Three-day total feces were collected during the treatment period from day 0-3 and from day 6-9. A: Data from total bile acid levels in the liver, small intestine, gallbladder, and total bile pool size and bile volume and concentration. B: Total bile acid levels in the colon and feces from Day 0-3 and Day 6-9. Additionally, fecal cholesterol and free fatty acids were measured from Day 6-9 fecal samples. All data represent mean±SEM. N=7-8 mice per group.

FIGS. 4A-4B: The Effects of Chronic long-acting FGF21 Analog Dosing on plasma total bile acids and C4 levels in Obese Cynomolgous Monkeys. A dose-escalation study was conducted in monkeys dosed weekly with Vehicle, AMG 875 (SEQ ID NO: 4), or AMG 876 (SEQ ID NO: 3). Monkeys were treated at 0.3 mg/kg for three weeks, followed by 3 weeks at 1 mg/kg dose, and another 3 weeks at 3 mg/kg. Plasma samples from overnight fasted monkeys were analyzed for plasma total bile acids and C4 levels. All data represent mean±SEM. N=10-14 monkeys per group.

### DETAILED DESCRIPTION OF THE INVENTION

Binding agents suitable for treating bile acid related disorders that activate the FGF21 signaling pathway can be prepared using the methods disclosed herein. Optionally, the half-life can be extended by fusing an antibody, or portion thereof, to the N-terminal or C-terminal end of the wild-type FGF21 sequence. It is also possible to further extend the half-life or decrease aggregation of the wild-type FGF21 protein by introducing amino acid substitutions into the protein. Such modified proteins are referred to herein as mutants, or FGF21 mutants, and form embodiments of the present invention. Further FGF21 pathway activating polypeptides include agonist antibodies.

### 1. General Definitions

The term “isolated nucleic acid molecule” refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “isolated nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecules or other contaminants that are found in its natural environment that would interfere with its use in polypeptide production or its therapeutic, diagnostic, prophylactic or research use.

Recombinant nucleic acid methods used herein, including in the Examples, are generally those set forth in Sambrook et al., Molecular Cloning: A Laboratory Manual (Cold Spring Harbor Laboratory Press, 1989) or Current Protocols in Molecular Biology (Ausubel et al., eds., Green Publishers Inc. and Wiley and Sons 1994).

The term “vector” is used to refer to any molecule (e.g., nucleic acid, plasmid, or virus) used to transfer coding information to a host cell.

The term “expression vector” refers to a vector that is suitable for transformation of a host cell and contains nucleic acid sequences that direct and/or control the expression of inserted heterologous nucleic acid sequences. Expression includes, but is not limited to, processes such as transcription, translation, and RNA splicing, if introns are present.

The term “operably linked” is used herein to refer to an arrangement of flanking sequences wherein the flanking sequences so described are configured or assembled so as to perform their usual function. Thus, a flanking sequence operably linked to a coding sequence may be capable of effecting the replication, transcription and/or translation of the coding sequence. For example, a coding sequence is operably linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “host cell” is used to refer to a cell which has been transformed, or is capable of being transformed with a nucleic acid sequence and then of expressing a selected gene of interest. The term includes the progeny of the parent cell, whether or not the progeny is identical in morphology or in genetic make-up to the original parent, so long as the selected gene is present.

The term “isolated polypeptide” refers to a polypeptide of the present invention that (1) has been separated from at least about 50 percent of polynucleotides, lipids, carbohydrates, or other materials with which it is naturally found when isolated from the source cell. (2) is not linked (by covalent or noncovalent interaction) to all or a portion of a polypeptide to which the “isolated polypeptide” is linked in nature, (3) is operably linked (by covalent or noncovalent interaction) to a polypeptide with which it is not linked in nature, or (4) does not occur in nature. Preferably, the isolated polypeptide is substantially free from any other contaminating polypeptides or other contaminants that are found in its natural environment that would interfere with its therapeutic, diagnostic, prophylactic or research use.

The term “naturally occurring” when used in connection with biological materials such as nucleic acid molecules, polypeptides, host cells, and the like, refers to materials which are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 amino acids alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y).

The term “FGF21 polypeptide” refers to a naturally-occurring wild-type polypeptide expressed in humans. For purposes of this disclosure, the term “FGF21 polypeptide” can be used interchangeably to refer to any full-length FGF21 polypeptide, e.g., SEQ ID NO: 12, which consists of 208 amino acid residues and which is encoded by the nucleotide sequence of SEQ ID NO: 13, any mature form of the polypeptide, e.g., SEQ ID NO: 1, which consists of 181 amino acid residues and which is encoded by the nucleotide sequence of SEQ ID NO: 2, and in which the 27 amino acid residues at the amino-terminal end of the full-length FGF21 polypeptide (i.e., which constitute the signal peptide) have been removed, and variants thereof.

The terms “FGF21 polypeptide mutant” and “FGF21 mutant” refer to an FGF21 polypeptide variant in which a naturally occurring FGF21 amino acid sequence has been modified. Such modifications include, but are not limited to, one or more amino acid substitutions, including substitutions with non-naturally occurring amino acid analogs, and truncations. Thus, FGF21 polypeptide mutants include, but are not limited to, site-directed FGF21 mutants, truncated FGF21 polypeptides, proteolysis-resistant FGF21 mutants, aggregation-reducing FGF21 mutants. FGF21 combination mutants, and FGF21 fusion proteins, as described herein. For the purpose of identifying the specific truncations and amino acid substitutions of the FGF21 mutants of the present invention, the numbering of the amino acid residues truncated or mutated corresponds to that of the mature 181-residue FGF21 polypeptide.

In other embodiments of the present invention, an FGF21 polypeptide mutant comprises an amino acid sequence that is at least about 85 percent identical to the amino acid sequence of SEQ ID NO: 1, but wherein specific residues conferring a desirable property to the FGF21 polypeptide mutant, e.g., proteolysis-resistance, increased half life or aggregation-reducing properties and combinations thereof, have not been further modified. In other words, with the exception of residues in the FGF21 mutant sequence that have been modified in order to confer proteolysis-resistance, aggregation-reducing, or other properties, about 15 percent of all other amino acid residues in the FGF21 mutant sequence can be modified. For example, in the FGF21 mutant Q173E, up to 15 percent of all amino acid residues other than the glutamic acid residue, which was substituted for glutamine at position 173, could be modified. In still other embodiments, an FGF21 polypeptide mutant comprises an amino acid sequence that is at least about 90 percent, or about 95, 96, 97, 98, or 99 percent identical to the amino acid sequence of SEQ ID NO: 1, but wherein the specific residues conferring the FGF21 polypeptide mutant's proteolysis-resistance or aggregation-reducing properties have not been further modified. Such FGF21 polypeptide mutants possess at least one activity of the wild-type FGF21 polypeptide.

The present invention also encompasses a nucleic acid molecule encoding an FGF21 polypeptide mutant comprising an amino acid sequence that is at least about 85 percent identical to the amino acid sequence of SEQ ID NO: 1, but wherein specific residues conferring a desirable property to the FGF21 polypeptide mutant, e.g., proteolysis-resistance, increased half life or aggregation-reducing properties and combinations thereof have not been further modified. In other words, with the exception of nucleotides that encode residues in the FGF21 mutant sequence that have been modified in order to confer proteolysis-resistance, aggregation-reducing, or other properties, about 15 percent of all other nucleotides in the FGF21 mutant sequence can be modified. For example, in the FGF21 mutant Q173E, up to 15 percent of all nucleotides other than the nucleotides encoding the glutamic acid residue, which was substituted for glutamine at position 173, could be modified. The present invention further encompasses a nucleic acid molecule encoding an FGF21 polypeptide mutant comprising an amino acid sequence that is at least about 90 percent, or about 95, 96, 97, 98, or 99 percent identical to the amino acid sequence of SEQ ID NO: 1, but wherein the specific residues conferring the FGF21 polypeptide mutant's proteolysis-resistance or aggregation-reducing properties have not been further modified. Such FGF21 mutants possess at least one activity of the wild-type FGF21 polypeptide.

The present invention also encompasses a nucleic acid molecule comprising a nucleotide sequence that is at least about 85 percent identical to the nucleotide sequence of SEQ ID NO: 2, but wherein the nucleotides encoding amino acid residues conferring the encoded FGF21 polypeptide mutant's proteolysis-resistance, aggregation-reducing or other properties have not been further modified. In other words, with the exception of residues in the FGF21 mutant sequence that have been modified in order to confer proteolysis-resistance, aggregation-reducing, or other properties, about 15 percent of all other amino acid residues in the FGF21 mutant sequence can be modified. For example, in the FGF21 mutant Q173E, up to 15 percent of all amino acid residues other than the glutamic acid residue, which was substituted for glutamine at position 173, could be modified. The present invention further encompasses a nucleic acid molecule comprising a nucleotide sequence that is at least about 90 percent, or about 95, 96, 97, 98, or 99 percent identical to the nucleotide sequence of SEQ ID NO: 2, but wherein the nucleotides encoding amino acid residues conferring the encoded FGF21 polypeptide mutant's proteolysis-resistance or aggregation-reducing properties have not been further modified. Such nucleic acid molecules encode FGF21 mutant polypeptides possessing at least one activity of the wild-type FGF21 polypeptide.

The term “biologically active FGF21 polypeptide mutant” refers to any FGF21 polypeptide mutant described herein that possesses an activity of the wild-type FGF21 polypeptide, such as the ability to lower blood glucose, insulin, triglyceride, or cholesterol; reduce body weight; and improve glucose tolerance, energy expenditure, or insulin sensitivity, regardless of the type or number of modifications that have been introduced into the FGF21 polypeptide mutant. FGF21 polypeptide mutants possessing a somewhat decreased level of FGF21 activity relative to the wild-type FGF21 polypeptide can nonetheless be considered to be biologically active FGF21 polypeptide mutants.

The terms “effective amount” and “therapeutically effective amount” each refer to the amount of an FGF21 polypeptide mutant used to support an observable level of one or more biological activities of the wild-type FGF21 polypeptide, such as the ability to lower blood glucose, insulin, triglyceride, or cholesterol levels; reduce body weight; or improve glucose tolerance, energy expenditure, or insulin sensitivity.

The term “pharmaceutically acceptable carrier” or “physiologically acceptable carrier” as used herein refers to one or more formulation materials suitable for accomplishing or enhancing the delivery of an FGF21 polypeptide mutant.

The term “antigen” refers to a molecule or a portion of a molecule that is capable of being bound by an antibody, and additionally that is capable of being used in an animal to produce antibodies that are capable of binding to an epitope of that antigen. An antigen may have one or more epitopes.

The term “native Fc” refers to molecule or sequence comprising the sequence of a non-antigen-binding fragment resulting from digestion of whole antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG (see Ellison et al., 1982, Nucleic Acids Res. 10: 4071-9). The term “native Fc” as used herein is generic to the monomeric, dimeric, and multimeric forms. An example of an Fc polypeptide sequence is presented in SEQ ID NO: 11.

The term “Fc variant” refers to a molecule or sequence that is modified from a native Fc but still comprises a binding site for the salvage receptor, FcRn (neonatal Fc receptor). International Publication Nos. WO 97/34631 and WO 96/32478 describe exemplary Fc variants, as well as interaction with the salvage receptor, and are hereby incorporated by reference. Thus, the term “Fc variant” can comprise a molecule or sequence that is humanized from a non-human native Fc. Furthermore, a native Fc comprises regions that can be removed because they provide structural features or biological activity that are not required for the fusion molecules of the FGF21 mutants of the present invention. Thus, the term “Fc variant” comprises a molecule or sequence that lacks one or more native Fc sites or residues, or in which one or more Fc sites or residues has be modified, that affect or are involved in: (1) disulfide bond formation. (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variants and sequences as defined above. As with Fc variants and native Fc molecules, the term “Fc domain” includes molecules in monomeric or multimeric form, whether digested from whole antibody or produced by other means. In some embodiments of the present invention, an Fc domain can be fused to FGF21 or a FGF21 mutant (including a truncated form of FGF21 or a FGF21 mutant) via, for example, a covalent bond between the Fc domain and the FGF21 sequence. Such fusion proteins can form multimers via the association of the Fc domains and both these fusion proteins and their multimers are an aspect of the present invention.

### 2. Site-Specific FGF21 Mutants

The term “site-specific FGF21 mutant” or “substituted FGF21 mutant” refers to an FGF21 mutant polypeptide having an amino acid sequence that differs from the amino acid sequence of a naturally occurring FGF21 polypeptide sequence, e.g., SEQ ID NOs: 1 and 14 and variants thereof. Site-specific FGF21 mutants can be generated by introducing amino acid substitutions, either conservative or non-conservative and using naturally or non-naturally occurring amino acids, at particular positions of the FGF21 polypeptide.

“Conservative amino acid substitution” can involve a substitution of a native amino acid residue (i.e., a residue found in a given position of the wild-type FGF21 polypeptide sequence) with a nonnative residue (i.e., a residue that is not found in a given position of the wild-type FGF21 polypeptide sequence) such that there is little or no effect on the polarity or charge of the amino acid residue at that position. Conservative amino acid substitutions also encompass non-naturally occurring amino acid residues that are typically incorporated by chemical peptide synthesis rather than by synthesis in biological systems. These include peptidomimetics, and other reversed or inverted forms of amino acid moieties.

Naturally occurring residues can be divided into classes based on common side chain properties:

(1) hydrophobic: norleucine, Met, Ala, Val, Leu, Ile;

(2) neutral hydrophilic: Cys, Ser, Thr;

(3) acidic: Asp, Glu;

(4) basic: Asn, Gin, His, Lys, Arg;

(5) residues that influence chain orientation: Gly, Pro; and

(6) aromatic: Trp, Tyr, Phe.

Conservative substitutions can involve the exchange of a member of one of these classes for another member of the same class. Non-conservative substitutions can involve the exchange of a member of one of these classes for a member from another class.

Desired amino acid substitutions (whether conservative or non-conservative) can be determined by those skilled in the art at the time such substitutions are desired. An exemplary (but not limiting) list of amino acid substitutions is set forth in Table 1.

TABLE 1
Amino Acid Substitutions

| Original Residue | Exemplary Substitutions |
|---|---|
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, Phe |
| Leu | Ile, Val, Met, Ala, Phe |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala |

### 3. Truncated FGF21 Polypeptides

One embodiment of the present invention is directed to truncated forms of the mature FGF21 polypeptide. This embodiment of the present invention arose from an effort to identify truncated FGF21 polypeptides that are capable of providing an activity that is similar, and in some instances superior, to untruncated forms of the mature FGF21 polypeptide.

As used herein, the term “truncated FGF21 polypeptide” refers to an FGF21 polypeptide in which amino acid residues have been removed from the amino-terminal (or N-terminal) end of the FGF21 polypeptide, amino acid residues have been removed from the carboxyl-terminal (or C-terminal) end of the FGF21 polypeptide, or amino acid residues have been removed from both the amino-terminal and carboxyl-terminal ends of the FGF21 polypeptide.

The activity of N-terminally truncated FGF21 polypeptides and C-terminally truncated FGF21 polypeptides can be assayed using an in vitro ELK-luciferase assay.

The activity of the truncated FGF21 polypeptides of the present invention can also be assessed in an in vivo assay, such as ob/ob mice. Generally, to assess the in vivo activity of a truncated FGF21 polypeptide, the truncated FGF21 polypeptide can be administered to a test animal intraperitoneally. After a desired incubation period (e.g., one hour or more), a blood sample can be drawn, and blood glucose levels can be measured.

a. N-terminal Truncations

In some embodiments of the present invention, N-terminal truncations comprise 1, 2, 3, 4, 5, 6, 7, or 8 amino acid residues from the N-terminal end of the mature FGF21 polypeptide. Truncated FGF21 polypeptides having N-terminal truncations of fewer than 9 amino acid residues retain the ability of the mature FGF21 polypeptide to lower blood glucose in an individual. Accordingly, in particular embodiments, the present invention encompasses truncated forms of the mature FGF21 polypeptide or FGF21 polypeptide mutants having N-terminal truncations of 1, 2, 3, 4, 5, 6, 7, or 8 amino acid residues.

b. C-Terminal Truncations

In some embodiments of the present invention, C-terminal truncations comprise 1, 2, 3, 4, 5, 6, 7, 8, 9, 10, 11, or 12 amino acid residues from the C-terminal end of the mature FGF21 polypeptide. Truncated FGF21 polypeptides having C-terminal truncations of fewer than 13 amino acid residues exhibited an efficacy of at least 50% of the efficacy of wild-type FGF21 in an in vitro ELK-luciferase assay, indicating that these FGF21 mutants retain the ability of the mature FGF21 polypeptide to lower blood glucose in an individual. Accordingly, in particular embodiments, the present invention encompasses truncated forms of the mature FGF21 polypeptide or FGF21 polypeptide mutants having C-terminal truncations of 1, 2, 3, 4, 5, 6, 7, 8, 9, 10, 11, or 12 amino acid residues.

### 4. Proteolysis-resistant FGF21 Mutants

Mature FGF21 was found to be undergoing in vivo degradation, which was ultimately determined to arise from proteolytic attack. The in vivo degradation of mature FGF21 was found to lead to shorter effective half-life, which can adversely affect the therapeutic potential of a molecule. Accordingly, a directed study was performed to identify FGF21 mutants that exhibit a resistance to proteolysis. As a result of this investigation, the sites in the mature FGF21 polypeptide that were determined to be particularly susceptible to proteolysis include the peptide bond between the amino acid residues at positions 4-5, 20-21, 151-152, and 171-172.

A broad but focused and directed study was performed to identify particular substitutions that eliminate the observed proteolytic effect while not affecting the activity of the protein to an unacceptable degree. Tables 8 and 11 highlight some of the mutants that were prepared and tested. Not all FGF21 mutants exhibited an ideal profile; some mutants conferred proteolysis resistance but at the cost of compromised FGF21 activity. Other mutations retained FGF21 activity but did not confer proteolysis resistance. Several mutants, including, for example, FGF21 P171G, retained a similar level of activity as wild-type FGF21 while also exhibiting resistance to proteolytic degradation.

One selection criteria for identifying desirable proteolysis-resistant FGF21 mutants was that the activity of the FGF21 mutant be essentially the same as, or greater than, the activity of wild-type FGF21. Therefore, another embodiment of the present invention is directed to FGF21 mutants that are resistant to proteolysis and still retain activity that is essentially the same as, or greater than, wild-type FGF21. Although less desirable in some cases. FGF21 mutants that are resistant to proteolysis but exhibit somewhat decreased activity form another embodiment of the present invention. In some cases it can be desirable to maintain a degree of proteolysis, and consequently, FGF21 mutants that allow some degree of proteolysis to occur also form another embodiment of the present invention.

As with all FGF21 mutants of the present invention, the proteolysis-resistant FGF21 mutants of the present invention can be prepared as described herein. Those of ordinary skill in the art, for example, those familiar with standard molecular biology techniques, can employ that knowledge, coupled with the instant disclosure, to make and use the proteolysis-resistant FGF21 mutants of the present invention. Standard techniques can be used for recombinant DNA, oligonucleotide synthesis, tissue culture, and transformation (e.g., electroporation, lipofection). See, e.g., Sambrook et al., Molecular Cloning: A Laboratory Manual, supra, which is incorporated herein by reference for any purpose. Enzymatic reactions and purification techniques can be performed according to manufacturer's specifications, as commonly accomplished in the art, or as described herein. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

The proteolysis-resistant FGF21 mutants of the present invention can be fused to another entity, which can impart additional properties to the proteolysis-resistant FGF21 mutant. In one embodiment of the present invention, a proteolysis-resistant FGF21 mutant can be fused to an IgG Fc sequence, e.g., SEQ ID NO: 11. Such fusion can be accomplished using known molecular biological methods and/or the guidance provided herein. The benefits of such fusion polypeptides, as well as methods for making such fusion polypeptides, are known and are discussed in more detail herein.

### 5. Aggregation-Reducing FGF21 Mutants

One property of the wild-type FGF21 polypeptide is its propensity to aggregate. At concentrations over about 5 mg/mL, the aggregation rate is high at room temperature. As shown and described herein, the aggregation rate for the wild-type FGF21 polypeptide is both concentration and temperature dependent.

Aggregation can prove to be a challenge when working with wild-type FGF21 at these concentrations, such as in the context of a therapeutic formulation. Accordingly, a directed study was performed to identify FGF21 mutants that exhibit reduced FGF21 aggregation. The resulting FGF21 mutants were then tested for the propensity to aggregate at various concentrations.

A broad but focused and directed study was performed to identify particular substitutions that eliminate or reduce the observed aggregation effect of wild-type FGF21 while not affecting the activity of the protein to an unacceptable degree.

One selection criteria for identifying desirable aggregation-reducing FGF21 mutants was that the activity of the FGF21 mutant be essentially similar to, or greater than, the activity of wild-type FGF21. Therefore, another embodiment of the present invention is directed to FGF21 mutants having reduced aggregation properties while still retaining an FGF21 activity that is similar to, or greater than, wild-type FGF21. Although less desirable in some cases, FGF21 mutants having reduced aggregation properties but exhibiting somewhat decreased FGF21 activity form another embodiment of the present invention. In some cases it may be desirable to maintain a degree of aggregation, and consequently, FGF21 mutants that allow some degree of aggregation to occur also form another embodiment of the present invention.

As with all FGF21 mutants of the present invention, the aggregation-reducing FGF21 mutants of the present invention can be prepared as described herein. Those of ordinary skill in the art, familiar with standard molecular biology techniques, can employ that knowledge, coupled with the instant disclosure, to make and use the aggregation-reducing FGF21 mutants of the present invention. Standard techniques can be used for recombinant DNA, oligonucleotide synthesis, tissue culture, and transformation (e.g., electroporation, lipofection). See, e.g., Sambrook et al., Molecular Cloning: A Laboratory Manual, supra, which is incorporated herein by reference for any purpose. Enzymatic reactions and purification techniques can be performed according to manufacturer's specifications, as commonly accomplished in the art, or as described herein. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques can be used for chemical syntheses; chemical analyses; pharmaceutical preparation, formulation, and delivery; and treatment of patients.

The aggregation-reducing FGF21 mutants of the present invention can be fused to another entity, which can impart additional properties to the aggregation-reducing FGF21 mutant. In one embodiment of the present invention, an aggregation-reducing FGF21 mutant can be fused to an IgG Fc sequence, e.g., SEQ ID NO: 11. Such fusion can be accomplished using known molecular biological methods and/or the guidance provided herein. The benefits of such fusion polypeptides, as well as methods for making such fusion polypeptides, are discussed in more detail herein.

### 6. FGF21 Fusion Proteins

As used herein, the term “FGF21 fusion polypeptide” or “FGF21 fusion protein” refers to a fusion of one or more amino acid residues (such as a heterologous protein or peptide) at the N-terminus or C-terminus of any FGF21 polypeptide mutant described herein.

Heterologous peptides and polypeptides include, but are not limited to, an epitope to allow for the detection and/or isolation of an FGF21 polypeptide mutant; a transmembrane receptor protein or a portion thereof, such as an extracellular domain or a transmembrane and intracellular domain; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region; a functional or non-functional antibody, or a heavy or light chain thereof; and a polypeptide which has an activity, such as a therapeutic activity, different from the FGF21 polypeptide mutants of the present invention. Also encompassed by the present invention are FGF21 mutants fused to human serum albumin (HSA).

Long acting FGF21 fusion proteins suitable for treating bile acid related disorders can be made by fusing heterologous sequences at either the N-terminus or at the C-terminus of an FGF21 polypeptide mutant. As described herein, a heterologous sequence can be an amino acid sequence or a non-amino acid-containing polymer. Heterologous sequences can be fused either directly to the FGF21 polypeptide mutant or via a linker or adapter molecule. A linker or adapter molecule can be one or more amino acid residues (or -mers), e.g., 1, 2, 3, 4, 5, 6, 7, 8, or 9 residues (or -mers), preferably from 10 to 50 amino acid residues (or -mers), e.g., 10, 11, 12, 13, 14, 15, 16, 17, 18, 19, 20, 25, 30, 35, 40, 45, or 50 residues (or -mers), and more preferably from 15 to 35 amino acid residues (or -mers). A linker or adapter molecule can also be designed with a cleavage site for a DNA restriction endonuclease or for a protease to allow for the separation of the fused moieties.

a. Fc Fusions

In one embodiment of the present invention, an FGF21 polypeptide mutant is fused to one or more domains of an Fc region of human IgG. Antibodies comprise two functionally independent parts, a variable domain known as “Fab,” that binds an antigen, and a constant domain known as “Fc,” that is involved in effector functions such as complement activation and attack by phagocytic cells. An Fc has a long serum half-life, whereas a Fab is short-lived (Capon et al., 1989, Nature 337: 525-31). When joined together with a therapeutic protein, an Fc domain can provide longer half-life or incorporate such functions as Fc receptor binding, protein A binding, complement fixation, and perhaps even placental transfer (Capon et al., 1989).

In vivo pharmacokinetic analysis indicated that human FGF21 has a short half-life of about 1 hour in mice due to rapid clearance and in vive degradation. Therefore, to extend the half-life of FGF21 an Fc sequence was fused to the N- or C-terminal end of the FGF21 polypeptide. The fusion of an Fc region to wild type FGF21, in particularly Fc fused to the N-terminus of wild type FGF21, did not extend the half-life as expected, however, which led to an investigation of the proteolytic degradation of FGF21 in vivo and the identification of FGF21 mutants that were resistant to such degradation. Such mutants exhibit longer half-lives than wild-type FGF21. These and other FGF21 fusion proteins form embodiments of the present invention.

Throughout the disclosure, Fc-FGF21 refers to a fusion protein in which the Fc sequence is fused to the N-terminus of FGF21. Similarly, throughout the disclosure, FGF21-Fc refers to a fusion protein in which the Fc sequence is fused to the C-terminus of FGF21.

The resulting FGF21 fusion protein can be purified, for example, by the use of a Protein A affinity column. Peptides and proteins fused to an Fc region have been found to exhibit a substantially greater half-life in vivo than the unfused counterpart. Also, a fusion to an Fc region allows for dimerization/multimerization of the fusion polypeptide. The Fc region can be a naturally occurring Fc region, or can be altered to improve certain qualities, such as therapeutic qualities, circulation time, or reduced aggregation.

Useful modifications of protein therapeutic agents by fusion with the “Fc” domain of an antibody are discussed in detail in International Publication No. WO 00/024782, which is hereby incorporated by reference in its entirety. This document discusses linkage to a “vehicle” such as polyethylene glycol (PEG), albumin, dextran, or an Fc region.

b. Fusion Protein Linkers

When forming the fusion proteins of the present invention, a linker can, but need not, be employed. When present, the linker's chemical structure may not critical, since it serves primarily as a spacer. The linker can be made up of amino acids linked together by peptide bonds. In some embodiments of the present invention, the linker is made up of from 1 to 20 amino acids linked by peptide bonds, wherein the amino acids are selected from the 20 naturally occurring amino acids. In various embodiments, the 1 to 20 amino acids are selected from the amino acids glycine, serine, alanine, proline, asparagine, glutamine, and lysine. In some embodiments, a linker is made up of a majority of amino acids that are sterically unhindered, such as glycine and alanine. In some embodiments, linkers are polyglycines (such as (Gly)₄ and (Gly)₅), polyalanines, combinations of glycine and alanine (such as poly(Gly-Ala)), or combinations of glycine and serine (such as poly(Gly-Ser)). Other suitable linkers include: (Gly)₅-Ser-(Gly)₃-Ser-(Gly)₄-Ser (SEQ ID NO: 5), (Gly)₄-Ser-(Gly)₄-Ser-(Gly)₄-Ser (SEQ ID NO: 6), (Gly)₃-Lys-(Gly)₄ (SEQ ID NO: 7), (Gly)₃-Asn-Gly-Ser-(Gly)₂ (SEQ ID NO: 8), (Gly)₃-Cys-(Gly)₄ (SEQ ID NO: 9), and Gly-Pro-Asn-Gly-Gly (SEQ ID NO: 10). While a linker of 15 amino acid residues has been found to work particularly well for certain FGF21 fusion proteins, the present invention contemplates linkers of suitable length or composition as determined by one of skill in the art.

The linkers described herein are exemplary, and linkers that are much longer and which include other residues are contemplated by the present invention. Non-peptide linkers are also contemplated by the present invention. For example, alkyl linkers such as —NH—(CH₂)ₛ—C(O)—, wherein s=2 to 20, could be used. These alkyl linkers can further be substituted by any non-sterically hindering group, including, but not limited to, a lower alkyl (e.g., C1-C6), lower acyl, halogen (e.g., Cl, Br), CN, NH₂, or phenyl. An exemplary non-peptide linker is a polyethylene glycol linker, wherein the linker has a molecular weight of 100 to 5000 kD, for example, 100 to 500 kD.

### 7. Chemically-Modified FGF21 Mutants

Chemically modified forms of the FGF21 polypeptide mutants described herein, including the truncated forms of FGF21 described herein, can be prepared by one skilled in the art, given the disclosures described herein. Such chemically modified FGF21 mutants are altered such that the chemically modified FGF21 mutant is different from the unmodified FGF21 mutant, either in the type or location of the molecules naturally attached to the FGF21 mutant. Chemically modified FGF21 mutants can include molecules formed by the deletion of one or more naturally-attached chemical groups.

In one embodiment, FGF21 polypeptide mutants of the present invention can be modified by the covalent attachment of one or more polymers. For example, the polymer selected is typically water-soluble so that the protein to which it is attached does not precipitate in an aqueous environment, such as a physiological environment. Included within the scope of suitable polymers is a mixture of polymers. Preferably, for therapeutic use of the end-product preparation, the polymer will be pharmaceutically acceptable. Non-water soluble polymers conjugated to FGF21 polypeptide mutants of the present invention also form an aspect of the invention.

Exemplary polymers each can be of any molecular weight and can be branched or unbranched. The polymers each typically have an average molecular weight of between about 2 kDa to about 100 kDa (the term “about” indicating that in preparations of a water-soluble polymer, some molecules will weigh more and some less than the stated molecular weight). The average molecular weight of each polymer is preferably between about 5 kDa and about 50 kDa, more preferably between about 12 kDa and about 40 kDa, and most preferably between about 20 kDa and about 35 kDa.

Suitable water-soluble polymers or mixtures thereof include, but are not limited to, N-linked or O-linked carbohydrates, sugars, phosphates, polyethylene glycol (PEG) (including the forms of PEG that have been used to derivatize proteins, including mono-(C₁-C₁₀), alkoxy-, or aryloxy-polyethylene glycol), monomethoxy-polyethylene glycol, dextran (such as low molecular weight dextran of, for example, about 6 kD), cellulose, or other carbohydrate based polymers, poly-(N-vinyl pyrrolidone) polyethylene glycol, propylene glycol homopolymers, polypropylene oxide/ethylene oxide co-polymers, polyoxyethylated polyols (e.g., glycerol), and polyvinyl alcohol. Also encompassed by the present invention are bifunctional crosslinking molecules that can be used to prepare covalently attached FGF21 polypeptide mutant multimers. Also encompassed by the present invention are FGF21 mutants covalently attached to polysialic acid.

In some embodiments of the present invention, an FGF21 mutant is covalently, or chemically, modified to include one or more water-soluble polymers, including, but not limited to, polyethylene glycol (PEG), polyoxyethylene glycol, or polypropylene glycol. See, e.g., U.S. Pat. Nos. 4,640,835; 4,496,689; 4,301.144; 4,670,417; 4,791,192; and 4,179,337. In some embodiments of the present invention, an FGF21 mutant comprises one or more polymers, including, but not limited to, monomethoxy-polyethylene glycol, dextran, cellulose, another carbohydrate-based polymer, poly-(N-vinyl pyrrolidone)-polyethylene glycol, propylene glycol homopolymers, a polypropylene oxide/ethylene oxide co-polymer, polyoxyethylated polyols (e.g., glycerol), polyvinyl alcohol, or mixtures of such polymers.

In some embodiments of the present invention, an FGF21 mutant suitable for treating bile acid related disorders is covalently-modified with PEG subunits. In some embodiments, one or more water-soluble polymers are bonded at one or more specific positions (for example, at the N-terminus) of the FGF21 mutant. In some embodiments, one or more water-soluble polymers are randomly attached to one or more side chains of an FGF21 mutant. In some embodiments, PEG is used to improve the therapeutic capacity of an FGF21 mutant. Certain such methods are discussed, for example, in U.S. Pat. No. 6,133,426, which is hereby incorporated by reference for any purpose.

In embodiments of the present invention wherein the polymer is PEG, the PEG group can be of any convenient molecular weight, and can be linear or branched. The average molecular weight of the PEG group will preferably range from about 2 kD to about 100 kDa, and more preferably from about 5 kDa to about 50 kDa, e.g., 10, 20, 30, 40, or 50 kDa. The PEG groups will generally be attached to the FGF21 mutant via acylation or reductive alkylation through a reactive group on the PEG moiety (e.g., an aldehyde, amino, thiol, or ester group) to a reactive group on the FGF21 mutant (e.g., an aldehyde, amino, or ester group).

The PEGylation of a polypeptide, including the FGF21 mutants of the present invention, can be specifically carried out using any of the PEGylation reactions known in the art. Such reactions are described, for example, in the following references: Francis et al., 1992, Focus on Growth Factors 3: 4-10; European Patent Nos. 0 154 316 and 0 401 384; and U.S. Pat. No. 4,179,337. For example, PEGylation can be carried out via an acylation reaction or an alkylation reaction with a reactive polyethylene glycol molecule (or an analogous reactive water-soluble polymer) as described herein. For the acylation reactions, a selected polymer should have a single reactive ester group. For reductive alkylation, a selected polymer should have a single reactive aldehyde group. A reactive aldehyde is, for example, polyethylene glycol propionaldehyde, which is water stable, or mono C₁-C₁₀ alkoxy or aryloxy derivatives thereof (see, e.g., U.S. Pat. No. 5,252,714).

In some embodiments of the present invention, a useful strategy for the attachment of the PEG group to a polypeptide involves combining, through the formation of a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Polysaccharide polymers are another type of water-soluble polymer that can be used for protein modification. Therefore, the FGF21 mutants of the present invention fused to a polysaccharide polymer form embodiments of the present invention. Dextrans are polysaccharide polymers comprised of individual subunits of glucose predominantly linked by alpha 1-6 linkages. The dextran itself is available in many molecular weight ranges, and is readily available in molecular weights from about 1 kD to about 70 kD. Dextran is a suitable water-soluble polymer for use as a vehicle by itself or in combination with another vehicle (e.g., Fc). See. e.g., International Publication No. WO 96/11953. The use of dextran conjugated to therapeutic or diagnostic immunoglobulins has been reported. See, e.g., European Patent Publication No. 0 315 456, which is hereby incorporated by reference. The present invention also encompasses the use of dextran of about 1 kD to about 20 kD.

In general, chemical modification can be performed under any suitable condition used to react a protein with an activated polymer molecule. Methods for preparing chemically modified polypeptides will generally comprise the steps of: (a) reacting the polypeptide with the activated polymer molecule (such as a reactive ester or aldehyde derivative of the polymer molecule) under conditions whereby a FGF21 polypeptide mutant becomes attached to one or more polymer molecules, and (b) obtaining the reaction products. The optimal reaction conditions will be determined based on known parameters and the desired result. For example, the larger the ratio of polymer molecules to protein, the greater the percentage of attached polymer molecule. In one embodiment of the present invention, chemically modified FGF21 mutants can have a single polymer molecule moiety at the amino-terminus (see, e.g., U.S. Pat. No. 5,234,784)

In another embodiment of the present invention, FGF21 polypeptide mutants can be chemically coupled to biotin. The biotin/FGF21 polypeptide mutants are then allowed to bind to avidin, resulting in tetravalent avidin/biotin/FGF21 polypeptide mutants. FGF21 polypeptide mutants can also be covalently coupled to dinitrophenol (DNP) or trinitrophenol (TNP) and the resulting conjugates precipitated with anti-DNP or anti-TNP-IgM to form decameric conjugates with a valency of 10.

Generally, conditions that can be alleviated or modulated by the administration of the present chemically modified FGF21 mutants include those described herein for FGF21 polypeptide mutants. However, the chemically modified FGF21 mutants disclosed herein can have additional activities, enhanced or reduced biological activity, or other characteristics, such as increased or decreased half-life, as compared to unmodified FGF21 mutants.

### 8. Therapeutic Compositions and Administration Thereof

Therapeutic compositions comprising FGF21 pathway activating molecules suitable for treating bile acid related disorders are within the scope of the present invention, and are specifically contemplated. Such pharmaceutical compositions can comprise a therapeutically effective amount of a polypeptide in admixture with a pharmaceutically or physiologically acceptable formulation agent selected for suitability with the mode of administration.

Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate. Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., Remington's Pharmaceutical Sciences (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage (see. e.g., Remington's Pharmaceutical Sciences, supra). Such compositions can influence the physical state, stability, rate of in vivo release, and rate of in vivo clearance of the FGF21 polypeptide mutant.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the present invention, FGF21 polypeptide mutant compositions can be prepared for storage by mixing the selected composition having the desired degree of purity with optional formulation agents (Remington's Pharmaceutical Sciences, supra) in the form of a lyophilized cake or an aqueous solution. Further, the FGF21 polypeptide mutant product can be formulated as a lyophilizate using appropriate excipients such as sucrose.

The pharmaceutical compositions can be selected for parenteral delivery. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use in this invention can be in the form of a pyrogen-free, parenterally acceptable, aqueous solution comprising the desired FGF21 polypeptide mutant in a pharmaceutically acceptable vehicle. A particularly suitable vehicle for parenteral injection is sterile distilled water in which an FGF21 polypeptide mutant is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

Additional pharmaceutical compositions will be evident to those skilled in the art, including formulations involving FGF21 polypeptide mutants in sustained- or controlled-delivery formulations. Techniques for formulating a variety of other sustained- or controlled-delivery means, such as liposome carriers, bio-erodible microparticles or porous beads and depot injections, are also known to those skilled in the art (see, e.g., International Publication No. WO 93/15722, which describes the controlled release of porous polymeric microparticles for the delivery of pharmaceutical compositions, and Wischke & Schwendeman, 2008, Int. J. Pharm. 364: 298-327, and Freiberg & Zhu, 2004, Int. J. Pharm. 282: 1-18, which discuss microsphere/microparticle preparation and use).

Additional examples of sustained-release preparations include semipermeable polymer matrices in the form of shaped articles, e.g. films, or microcapsules. Sustained release matrices can include polyesters, hydrogels, polylactides (U.S. Pat. No. 3,773,919 and European Patent No. 0 058 481), copolymers of L-glutamic acid and gamma ethyl-L-glutamate (Sidman et al., 1983, Biopolymers 22: 547-56), poly(2-hydroxyethyl-methacrylate) (Langer et al., 1981, J. Biomed. Mater. Res. 15: 167-277 and Langer, 1982, Chem. Tech. 12: 98-105), ethylene vinyl acetate (Langer et al., supra) or poly-D(−)-3-hydroxybutyric acid (European Patent No. 0 133 988). Sustained-release compositions can also include liposomes, which can be prepared by any of several methods known in the art. See, e.g., Epstein et al., 1985, Proc. Natl. Acad. Sci. U.S.A. 82: 3688-92; and European Patent Nos. 0 036 676, 0 088 046, and 0 143 949.

The pharmaceutical composition to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In a specific embodiment, the present invention is directed to kits for producing a single-dose administration unit. The kits can each contain both a first container having a dried protein and a second container having an aqueous formulation. Also included within the scope of this invention are kits containing single and multi-chambered pre-filled syringes (e.g., liquid syringes and lyosyringes).

The effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the FGF21 polypeptide mutant is being used, the route of administration, and the size (body weight, body surface, or organ size) and condition (the age and general health) of the patient. Accordingly, the clinician can titer the dosage and modify the route of administration to obtain the optimal therapeutic effect. A typical dosage can range from about 0.1 μg/kg to up to about 100 mg/kg or more, depending on the factors mentioned above. In other embodiments, the dosage can range from 0.1 μg/kg up to about 100 mg/kg; or 1 μg/kg up to about 100 mg/kg; or 5 μg/kg, 10 μg/kg, 15 μg/kg, 20 μg/kg, 25 μg/kg, 30 μg/kg, 35 g/kg, 40 μg/kg, 45 μg/kg, 50 μg/kg, 55 μg/kg, 60 μg/kg, 65 μg/kg, 70 μg/kg, 75 μg/kg, up to about 100 μg/kg. In yet other embodiments, the dosage can be 50 μg/kg, 100 μg/kg, 150 μg/kg, 200 μg/kg, 250 μg/kg, 300 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 100 μg/kg, 200 μg/kg, 300 μg/kg, 400 μg/kg, 500 μg/kg, 600 μg/kg, 700 μg/kg, 800 μg/kg, 900 μg/kg, 1000 μg/kg, 2000 μg/kg, 3000 μg/kg, 4000 μg/kg, 5000 μg/kg, 6000 μg/kg, 7000 μg/kg, 8000 μg/kg, 9000 μg/kg or 10 mg/kg.

The frequency of dosing will depend upon the pharmacokinetic parameters of the therapeutic in the formulation being used for treating the bile acid related disease or disorder. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., through injection by intravenous, intraperitoneal, intracerebral (intraparenchymal), intracerebroventricular, intramuscular, intraocular, intraarterial, intraportal, or intralesional routes; by sustained release systems (which may also be injected); or by implantation devices. Where desired, the compositions can be administered by bolus injection or continuously by infusion, or by implantation device.

Alternatively or additionally, the composition can be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

### 9. Therapeutic Uses of FGF21

FGF21 signaling proteins can be used to treat, diagnose, ameliorate, or prevent a number of diseases, disorders, or conditions, including, but not limited to disorders or conditions for which reduction of bile acid levels is desired and include progressive familial intrahepatic cholestasis type 2 and 3 (BSEP and MDR3 mutations respectively, these are pumps that export bile acids and phospholipid out of liver), intrahepatic cholestasis of pregnancy (ICP), drug-induced cholestasis, contraceptive-induced cholestasis, primary biliary cirrhosis (autoimmune), primary sclerosing cholangitis (autoimmune), cryptogenic biliary fibrosis/cirrhosis, total parenteral nutrition (TPN)-induced cholestasis, bile duct injury following liver transplantation, sepsis-associated cholestasis, progressive sclerosing cholangitis, idiopathic adulthood ductopenia, oriental cholangiohepatitis, and cholangiopathy associated with primary hepatolithiasis.

These diseases or disorders can be treated by administering a long acting FGF21 agonist as described herein to a patient in need thereof in the amount of a therapeutically effective dose. The administration can be performed as described herein, such as by IV injection, intraperitoneal injection, intramuscular injection in the form of a liquid formation or lyophilized. In most situations, a desired dosage can be determined by a clinician, as described herein, and can represent a therapeutically effective dose of the FGF21 mutant polypeptide. It will be apparent to those of skill in the art that a therapeutically effective dose of FGF21 mutant polypeptide will depend, inter alia, upon the administration schedule, the unit dose of antigen administered, whether the nucleic acid molecule or polypeptide is administered in combination with other therapeutic agents, the immune status and the health of the recipient.

The term “therapeutically effective dose,” as used herein, means that amount of FGF21 pathway activator that elicits the biological or medicinal response in a tissue system, animal, or human being sought by a researcher, medical doctor, or other clinician, which includes alleviation of the symptoms of the disease or disorder being treated.

### 10. Antibodies

Antibodies and antibody fragments that activate the FGF21 signaling pathway are contemplated and are within the scope of the present invention. The antibodies can be polyclonal, including monospecific polyclonal; monoclonal (MAbs); recombinant; chimeric; humanized, such as complementarity-determining region (CDR)-grafted; human; single chain; and/or bispecific; as well as fragments; variants; or chemically modified molecules thereof. Antibody fragments include those portions of the antibody that specifically bind to an epitope on an FGF21 mutant polypeptide. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies that mimic, agonize or activate the FGF21 signaling pathway can be produced using any method that provides for the production of antibody molecules by continuous cell lines in culture. Examples of suitable methods for preparing monoclonal antibodies include the hybridoma methods of Kohler et al., 1975, Nature 256: 495-97 and the human B-cell hybridoma method (Kozbor, 1984, J. Immunol. 133: 3001; Brodeur et al., Monoclonal Antibody Production Techniques and Applications 51-63 (Marcel Dekker, Inc., 1987). Also provided by the invention are hybridoma cell lines that produce monoclonal antibodies reactive with FGF21 mutant polypeptides.

Monoclonal antibodies of the invention can be modified for use as therapeutics. In one embodiment, the monoclonal antibody is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See, e.g., U.S. Pat. No. 4,816,567; Morrison et al., 1985, Proc. Natl. Acad. Sci. U.S.A. 81: 6851-55.

In another embodiment, a monoclonal antibody of the invention is a “humanized” antibody. Methods for humanizing non-human antibodies are well known in the art. See, e.g., U.S. Pat. Nos. 5,585,089 and 5,693,762. Generally, a humanized antibody has one or more amino acid residues introduced into it from a source that is non-human. Humanization can be performed, for example, using methods described in the art (see, e.g., Jones et al., 1986, Nature 321: 522-25; Riechmann et al., 1998. Nature 332: 323-27; Verhoeyen et al., 1988, Science 239: 1534-36), by substituting at least a portion of a rodent complementarity-determining region for the corresponding regions of a human antibody.

Also encompassed by the invention are human antibodies that bind the FGF21 mutant polypeptides of the present invention. Using transgenic animals (e.g., mice) that are capable of producing a repertoire of human antibodies in the absence of endogenous immunoglobulin production such antibodies are produced by immunization with an FGF21 mutant antigen (i.e., having at least 6 contiguous amino acids), optionally conjugated to a carrier. See, e.g., Jakobovits et al., 1993, Proc. Natl. Acad. Sci. U.S.A. 90: 2551-55; Jakobovits et al., 1993, Nature 362: 255-58; Bruggermann et al., 1993, Year in Immuno. 7: 33. In one method, such transgenic animals are produced by incapacitating the endogenous loci encoding the heavy and light immunoglobulin chains therein, and inserting loci encoding human heavy and light chain proteins into the genome thereof. Partially modified animals, i.e., animals having less than the full complement of modifications, are then cross-bred to obtain an animal having all of the desired immune system modifications. When administered an immunogen, these transgenic animals produce antibodies with human (rather than, e.g., murine) amino acid sequences, including variable regions that are immunospecific for these antigens. See, e.g., International Publication Nos. WO 96/33735 and WO 94/02602. Additional methods are described in U.S. Pat. No. 5,545,807, International Publication Nos. WO 91/10741 and WO 90/04036, and in European Patent No. 0 546 073. Human antibodies can also be produced by the expression of recombinant DNA in host cells or by expression in hybridoma cells as described herein.

In an alternative embodiment, human antibodies can also be produced from phage-display libraries (see, e.g., Hoogenboom et al., 1991, J. Mol. Biol. 227: 381; Marks et al., 1991, J. Mol. Biol. 222: 581). These processes mimic immune selection through the display of antibody repertoires on the surface of filamentous bacteriophage, and subsequent selection of phage by their binding to an antigen of choice. One such technique is described in International Publication No. WO 99/10494, which describes the isolation of high affinity and functional agonistic antibodies for MPL- and msk- receptors using such an approach.

Chimeric, CDR grafted, and humanized antibodies are typically produced by recombinant methods. Nucleic acids encoding the antibodies are introduced into host cells and expressed using materials and procedures described herein. In one embodiment, the antibodies are produced in mammalian host cells, such as CHO cells. Monoclonal (e.g., human) antibodies can be produced by the expression of recombinant DNA in host cells or by expression in hybridoma cells as described herein.

The antibodies of the invention can be employed in any known assay method, such as competitive binding assays, direct and indirect sandwich assays, and immunoprecipitation assays (see, e.g., Sola, Monoclonal Antibodies: A Manual of Techniques 147-158 (CRC Press, Inc., 1987), incorporated herein by reference in its entirety) for the detection and quantitation of FGF21 mutant polypeptides. The antibodies will bind FGF21 mutant polypeptides with an affinity that is appropriate for the assay method being employed.

For diagnostic applications, in certain embodiments, antibodies can be labeled with a detectable moiety. The detectable moiety can be any one that is capable of producing, either directly or indirectly, a detectable signal. For example, the detectable moiety can be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, fi-galactosidase, or horseradish peroxidase (Bayer et al., 1990, Meth. Enz. 184: 138-63).

Competitive binding assays rely on the ability of a labeled standard (e.g., an FGF21 mutant polypeptide, or an immunologically reactive portion thereof) to compete with the test sample analyte (e.g., an FGF21 mutant polypeptide) for binding with a limited amount of anti-FGF21 mutant antibody. The amount of an FGF21 mutant polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies can conveniently be separated from the standard and analyte that remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody that is immobilized on a solid support, and thereafter a second antibody binds to the analyte, thus forming an insoluble three-part complex. See, e.g., U.S. Pat. No. 4,376,110. The second antibody can itself be labeled with a detectable moiety (direct sandwich assays) or can be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

The antibodies of the present invention are also useful for in vive imaging. An antibody labeled with a detectable moiety can be administered to an animal, preferably into the bloodstream, and the presence and location of the labeled antibody in the host assayed. The antibody can be labeled with any moiety that is detectable in an animal, whether by nuclear magnetic resonance, radiology, or other detection means known in the art.

The of the invention can be used as therapeutics. These therapeutic agents are generally agonists or antagonists, in that they either enhance or reduce, respectively, at least one of the biological activities of an FGF21 mutant polypeptide. In one embodiment, antagonist antibodies of the invention are antibodies or binding fragments thereof which are capable of specifically binding to an FGF21 mutant polypeptide and which are capable of inhibiting or eliminating the functional activity of an FGF21 mutant polypeptide in vivo or in vitro.

### EXAMPLES

The Examples that follow are illustrative of specific embodiments of the invention, and various uses thereof. They are set forth for explanatory purposes only, and should not be construed as limiting the scope of the invention in any way.

### Preparation of FGF21 Expression Constructs

A nucleic acid sequence encoding the mature FGF21 polypeptide was obtained by polymerase chain reaction (PCR) amplification using primers having nucleotide sequences corresponding to the 5′ and 3′ ends of the mature FGF21 sequence. Table 2 lists the primers that were used to amplify the mature FGF21 sequence.

TABLE 2
PCR Primers for Preparing EGF21 Construct

| | | SEQ |
|---|---|---|
| Primer | Sequence | ID NO: |
| Sense | 5′-AGGAGGAATAACATATGCATCCAATTCCAGA | 14 |
| | TTCTTCTCC-3′ | |
| Anti- | 5′-TAGTGAGCTCGAATTCTTAGGAAGCGTAGCT | 15 |
| sense | GG-3′ | |

The primers used to prepare the FGF21 expression construct incorporated restriction endonuclease sites for directional cloning of the sequence into a suitable expression vector (e.g., pET30 (Novagen/EMD Biosciences; San Diego, Calif.) or pAMG33 (Amgen; Thousand Oaks, Calif.)). The expression vector pAMG33 contains a low-copy number R-100 origin of replication, a modified lac promoter, and a kanamycin-resistance gene. The expression vector pET30 contains a pBR322-derived origin of replication, an inducible T7 promoter, and a kanamycin-resistance gene. While expression from pAMG33 was found to be higher, pET30 was found to be a more reliable cloning vector. Thus, the majority of the constructs described in the instant application were first generated in pET30 and then screened for efficacy. Selected sequences were then transferred to pAMG33 for further amplification.

The FGF21 sequence was amplified in a reaction mixture containing 40.65 μL dH₂O, 5 μL PfuUltra II Reaction Buffer (10×), 1.25 μL dNTP Mix (40 mM-4×10 mM), 0.1 μL Template (100 ng/mL), 1 μL Primer1 (10 μM), 1 μL Primer2 (10 μM), and 1 μL PfuUltra II fusion HS DNA Polymerase (Stratagene; La Jolla, Calif.). Amplification reactions were performed by heating for 2 minutes at 95° C.; followed by ten cycles at 95° C. for 20 seconds, 60° C. for 20 seconds (with an additional 1° C. subtracted per cycle), and 72° C. for 15 seconds/kilobase of desired product; followed by 20 cycles at 94° C. for 20 seconds, 55° C. for 20 seconds, and 72° C. for 15 seconds/kilobase of desired product; followed by 72° C. for 3 minutes. Amplification products were digested with the restriction endonucleases NdeI, DpnI, and EcoRI; ligated into a suitable vector; and then transformed into competent cells.

### Purification of FGF21 Proteins from Bacteria

In the Examples that follow, various FGF21 proteins, including the wild-type FGF21 polypeptide, truncated FGF21 polypeptides, FGF21 mutants, and FGF21 fusion proteins, were expressed in a bacterial expression system. After expression, which is described below, the FGF21 proteins were purified as described in this Example, unless otherwise indicated.

To purify the wild-type FGF21 polypeptide, truncated FGF21 polypeptides, and FGF21 mutants from bacterial inclusion bodies, double-washed inclusion bodies (DWIBs) were solubilized in a solubilization buffer containing guanidine hydrochloride and DTT in Tris buffer at pH 8.5 and then mixed for one hour at room temperature, and the solubilization mixture was added to a refold buffer containing urea, arginine, cysteine, and cystamine hydrochloride at pH 9.5 and then mixed for 24 hours at 5° C. (see, e.g., Clarke, 1998, Curr. Opin. Biotechnol. 9: 157-63; Mannall et al., 2007, Biotechnol. Bioeng. 97: 1523-34; Rudolph et al., 1997, “Folding proteins,” Protein Function: A Practical Approach (Creighton, ed., New York, IRL Press) 57-99; and Ishibashi et al., 2005, Protein Expr. Purif. 42: 1-6).

Following solubilization and refolding, the mixture was filtered through a 0.45 micron filter. The refold pool was then concentrated approximately 10-fold with a 10 kD molecular weight cut-off Pall Omega cassette at a transmembrane pressure (TMP) of 20 psi, and dialfiltered with 3 column volumes of 20 mM Tris, pH 8.0 at a TMP of 20 psi.

The clarified sample was then subjected to anion exchange (AEX) chromatography using a Q Sepharose HP resin. A linear salt gradient of 0 to 250 mM NaCl in 20 mM Tris was run at pH 8.0 at 5° C. Peak fractions were analyzed by SDS-PAGE and pooled.

The AEX eluate pool was then subjected to hydrophobic interaction chromatography (HIC) using a Phenyl Sepharose HP resin. Protein was eluted using a decreasing linear gradient of 0.7 M to 0 M ammonium sulfate at pH 8.0 and ambient temperature. Peak fractions were analyzed by SDS-PAGE (Laemmli, 1970, Nature 227: 680-85) and pooled.

The HIC pool was concentrated with a 10 kD molecular weight cut-off Pall Omega 0.2 m² cassette to 7 mg/mL at a TMP of 20 psi. The concentrate was dialfiltered with 5 column volumes of 10 mM KPO₄, 5% sorbitol, pH 8.0 at a TMP of 20 psi, and the recovered concentrate was diluted to 5 mg/mL. Finally, the solution was filtered through a Pall mini-Kleenpac 0.2 μM Posidyne membrane.

To purify FGF21 fusion proteins and FGF21 fusion mutant proteins from bacterial inclusion bodies, double-washed inclusion bodies (DWIBs) were solubilized in a solubilization buffer containing guanidine hydrochloride and DTT in Tris buffer at pH 8.5 and then mixed for one hour at room temperature, and the solubilization mixture was added to a refold buffer containing urea, arginine, cysteine, and cystamine hydrochloride at pH 9.5 and then mixed for 24 hours at 5° C. (see, e.g., Clarke, 1998, Curr. Opin. Biotechnol. 9: 157-63; Mannall et al., 2007, Biotechnol. Bioeng. 97: 1523-34; Rudolph et al., 1997, “Folding proteins,” Protein Function: A Practical Approach (Creighton, ed., New York, IRL Press) 57-99; and Ishibashi et al., 2005, Protein Expr. Purif 42: 1-6).

Following solubilization and refolding, the mixture was dialyzed against 5 volumes of 20 mM Tris, pH 8.0 using 10 kD dialysis tubing. The pH of the dialyzed refold was adjusted to 5.0 with 50% acetic acid, and then clarified by centrifugation for 30 minutes at 4K.

The clarified sample was then subjected to anion exchange (AEX) chromatography using a Q Sepharose HP resin. A linear salt gradient of 0 to 250 mM NaCl in 20 mM Tris was run at pH 8.0 at 5° C. Peak fractions were analyzed by SDS-PAGE (Laemmli, 1970. Nature 227: 680-85) and pooled.

The AEX eluate pool was then subjected to hydrophobic interaction chromatography (HIC) using a Phenyl Sepharose HP resin. Protein was eluted using a decreasing linear gradient of 0.6 M to 0 M ammonium sulfate at pH 8.0 at ambient temperature. Peak fractions were analyzed by SDS-PAGE and pooled.

Following the HIC step, the pool was then dialyzed 60 volumes of 10 mM Tris, 2.2% sucrose, 3.3% sorbitol, pH 8.5. The dialyzed pool was concentrated to 5 mg/mL using a jumbosep. Finally, the solution was filtered through a Pall mini-Kleenpac 0.2 μM Posidyne membrane.

### FGF21 Proteolysis-Resistant Mutants

Suitable FGF21 mutants were identified by experimentally determining the positions of the wild-type FGF21 sequence that are sites of major proteolytic activity, and specific amino acid substitutions were introduced at these sites. Amino acid substitutions were based on FGF21 sequence conservation with other species and biochemical conservation with other amino acid residues. A list of amino acid substitutions that were or can be introduced into the wild-type FGF21 protein is provided in Table 3, although Table 3 is only exemplary and other substitutions can be made. The numbers of the positions given in Table 3 correspond to the residue position in the mature FGF21 protein, which consists of 181 amino acid residues.

TABLE 3
FGF21 Residues Mutated

| Amino Acid | Native | |
|---|---|---|
| Position | Residue | Mutations |
| 19 | Arg | Gln, Ile, Lys |
| 20 | Tyr | His, Leu, Phe |
| 21 | Leu | Ile, Phe, Tyr, Val |
| 22 | Tyr | Ile, Phe, Val |
| 150 | Pro | Ala, Arg |
| 151 | Gly | Ala, Val |
| 152 | Ile | His, Leu, Phe, Val |
| 170 | Gly | Ala, Asn, Asp, Cys, Gln, Glu, Pro, Ser |
| 171 | Pro | Ala, Arg, Asn, Asp, Cys, Glu, Gln, Gly, |
| | | His, Lys, Ser, Thr, Trp, Tyr |
| 172 | Ser | Leu, Thr |
| 173 | Gln | Arg, Glu |

### Preparation and Expression of Proteolysis-Resistant FGF21

### Mutants and Fusion Proteins

Constructs encoding the FGF21 mutants were prepared by PCR amplification of the wild-type FGF21 expression vector as described below. The goal of these experiments was to generate FGF21 mutants that are resistant to proteolysis and exhibit longer half-lives.

TABLE 4
Proteolysis-Resistant FGF21 Mutants

| Mutation(s) | Fc | Linker |
|---|---|---|
| R19I | | |
| R19I | —COOH | 15 |
| R19K | | |
| R19K | —COOH | 15 |
| R19Q | | |
| R19Q | —COOH | 15 |
| R19K, Y20H | | |
| R19K, Y20H | —COOH | 15 |
| R19K, L21I | | |
| R19K, L21I | —COOH | 15 |
| R19K, Y20H, L21I | | |
| R19K, Y20H, L21I | —COOH | 15 |
| Y20F | | |
| Y20F | —COOH | 15 |
| Y20H | | |
| Y20H | —COOH | 15 |
| Y20L | | |
| Y20L | —COOH | 15 |
| Y20H, L21I | | |
| Y20H, L21I | —COOH | 15 |
| L21I | | |
| L21I | —COOH | 15 |
| L21F | | |
| L21F | —COOH | 15 |
| L21V | | |
| L21V | —COOH | 15 |
| L21Y | | |
| L21Y | —COOH | 15 |
| Y22F | | |
| Y22F | —COOH | 15 |
| Y22I | | |
| Y22I | —COOH | 15 |
| Y22V | | |
| Y22V | —COOH | 15 |
| P150A | | |
| P150A | —NH₂ | 15 |
| P150R | —NH₂ | 15 |
| P150A, G151A | | |
| P150A, G151A | —NH₂ | 15 |
| P150A, I152V | | |
| P150A, I152V | —NH₂ | 15 |
| P150A, G151A, I152V | | |
| P150A, G151A, I152V | —NH₂ | 15 |
| G151A | | |
| G151A | —NH₂ | 15 |
| G151V | | |
| G151V | —NH₂ | 15 |
| G151A, I152V | | |
| G151A, I152V | —NH₂ | 15 |
| I152F | | |
| I152F | —NH₂ | 15 |
| I152H | | |
| I152H | —NH₂ | 15 |
| I152L | | |
| I152L | —NH₂ | 15 |
| I152V | | |
| G170A | | |
| G170A | —NH₂ | 15 |
| G170C | | |
| G170C | —NH₂ | 15 |
| G170D | | |
| G170D | —NH₂ | 15 |
| G170E | | |
| G170E | —NH₂ | 15 |
| G170N | | |
| G170N | —NH₂ | 15 |
| G170P | | |
| G170P | —NH₂ | 15 |
| G170Q | | |
| G170Q | —NH₂ | 15 |
| G170S | | |
| G170S | —NH₂ | 15 |
| G170E, P171A | | |
| G170E, P171A | —NH₂ | 15 |
| G170E, S172L | | |
| G170E, S172L | —NH₂ | 15 |
| G170E, P171A, S172L | | |
| G170E, P171A, S172L | —NH₂ | 15 |
| P171A | | |
| P171A | —NH₂ | 15 |
| P171C | —NH₂ | 15 |
| P171D | —NH₂ | 15 |
| P171E | —NH₂ | 15 |
| P171G | —NH₂ | 15 |
| P171H | —NH₂ | 15 |
| P171K | —NH₂ | 15 |
| P171N | —NH₂ | 15 |
| P171Q | —NH₂ | 15 |
| P171S | —NH₂ | 15 |
| P171T | —NH₂ | 15 |
| P171W | —NH₂ | 15 |
| P171Y | —NH₂ | 15 |
| P171A, S172L | | |
| P171A, S172L | —NH₂ | 15 |
| S172L | —NH₂ | 15 |
| S172T | | |
| S172T | —NH₂ | 15 |
| Q173E | | |
| Q173E | —NH₂ | 15 |
| Q173R | | |
| Q173R | —NH₂ | 15 |

FGF21 mutant constructs were prepared using primers having sequences that are homologous to regions upstream and downstream of a codon (or codons) to be mutated. The primers used in such amplification reactions also provided approximately 15 nucleotides of overlapping sequence to allow for recircularization of the amplified product, namely the entire vector now having the desired mutant.

FGF21 mutant constructs were prepared using essentially the PCR conditions. Amplification products were digested with the restriction endonuclease DpnI, and then transformed into competent cells. The resulting clones were sequenced to confirm the absence of polymerase-generated errors. Fc-FGF21 and FGF21-Fc fusion proteins were generated as described herein.

FGF21 mutants were expressed by transforming competent BL21 (DE3) or BL21 Star (Invitrogen; Carlsbad, Calif.) cells with the construct encoding a particular mutant. Transformants were grown overnight with limited aeration in TB media supplemented with 40 μg/mL kanamycin, were aerated the next morning, and after a short recovery period, were induced in 0.4 mM IPTG. FGF21 mutant polypeptides were harvested by centrifugation 18-20 hours after induction.

FGF21 mutants were also analyzed for predicted immunogenicity. Immune responses against proteins are enhanced by antigen processing and presentation in the major histocompatability complex (MHC) class II binding site. This interaction is required for T cell help in maturation of antibodies that recognize the protein. Since the binding sites of MHC class II molecules have been characterized, it is possible to predict whether proteins have specific sequences that can bind to a series of common human alleles. Computer algorithms have been created based on literature references and MHC class II crystal structures to determine whether linear amino acid peptide sequences have the potential to break immune tolerance. The TEPITOPE computer program was used to determine if point mutations in particular FGF21 mutants would increase antigen specific T cells in a majority of humans. Based on an analysis of the linear protein sequence of each FGF21 mutant, none of the mutants was predicted to enhance immunogenicity.

### Preparation and Expression of Fc-FGF21 Fusion Combination Mutants

As described above, the stability and solubility of FGF21 can be modulated through the introduction of specific truncations and amino acid substitutions. In addition, FGF21 stability can be further enhanced by fusing such modified FGF21 proteins with the Fc portion of the human immunoglobulin IgG1 gene. Moreover, by introducing combinations of the above modifications, FGF21 molecules having both enhanced stability and solubility can be generated. Nucleic acid sequences encoding the FGF21 combination mutants listed in Table 6 were prepared using the techniques described above.

TABLE 6
FGF21 Combination Mutants

| Amino Acid | Proteolysis | Aggregation | | |
|---|---|---|---|---|
| Residues | Mutation | Mutation | Fc | Linker |
| 1-181 | G170E | A45K | —NH₂ | 15 |
| 1-181 | G170E | L98R | —NH₂ | 15 |
| 1-181 | G170E | A45K, L98R | —NH₂ | 15 |
| 1-181 | P171G | A45K | —NH₂ | 15 |
| 1-181 | P171S | A45K | —NH₂ | 15 |
| 1-181 | P171G | L98R | —NH₂ | 15 |
| 1-181 | P171S | L98R | —NH₂ | 15 |
| 1-181 | P171G | A45K, L98R | —NH₂ | 15 |
| 1-178 | G170E | | —NH₂ | 15 |
| 6-181 | G170E | | —NH₂ | 15 |
| 6-181 | G170E | A45K | —NH₂ | 15 |
| 6-181 | G170E | L98R | —NH₂ | 15 |
| 6-181 | P171G | | —NH₂ | 15 |
| 6-181 | P171G | L98R | —NH₂ | 15 |
| 7-181 | G170E | | —NH₂ | 15 |

### Acute Effect of Recombinant FGF21 on CYP7A1 Expression in Mice

Cholesterol 7 alpha-hydroxylase (CYP7A1) is the rate-limiting enzyme involved in bile acid synthesis via the classic pathway. A reduction in CYP7A1 gene expression would indicate a down-regulation of bile acid synthesis. Five separate studies were conducted to evaluate the effects of recombinant FGF21 on CYP7A1 expression in different mouse models after a single administration. All mice were allowed to acclimate to a 12:12-h light-dark cycle, housing humidity and temperature, and routine handling prior to initiation of each study. Lean male C57BL6 mice (Harlan Laboratories) were maintained on a standard rodent diet (2020× Harlan Teklad). For studies involving diet-induced obese (DIO) mice, male C57BL6 mice were obtained from Charles River Laboratories (Hollister, Calif.) at 3 weeks of age. Obesity was induced at 4 weeks of age by initiating a 606 kcal high-fat diet (D12492, Research Diets) feeding and continuing for at least 12 weeks prior to study initiation. DIO mice were maintained on the high-fat diet for the duration of each study. Leptin-deficient ob/ob mice were obtained from Jackson Laboratories (stock #000632) at 8 weeks of age, group-housed and maintained on a standard rodent diet (8640 Harlan Teklad). Mice from all three mouse models were stratified into treatment groups based on body weight. A single intraperitoneal injection (IP) of recombinant FGF21 was administered at indicated doses. Terminal blood and liver samples were collected at various time points post injection for measurement of drug concentration and to perform gene expression analysis.

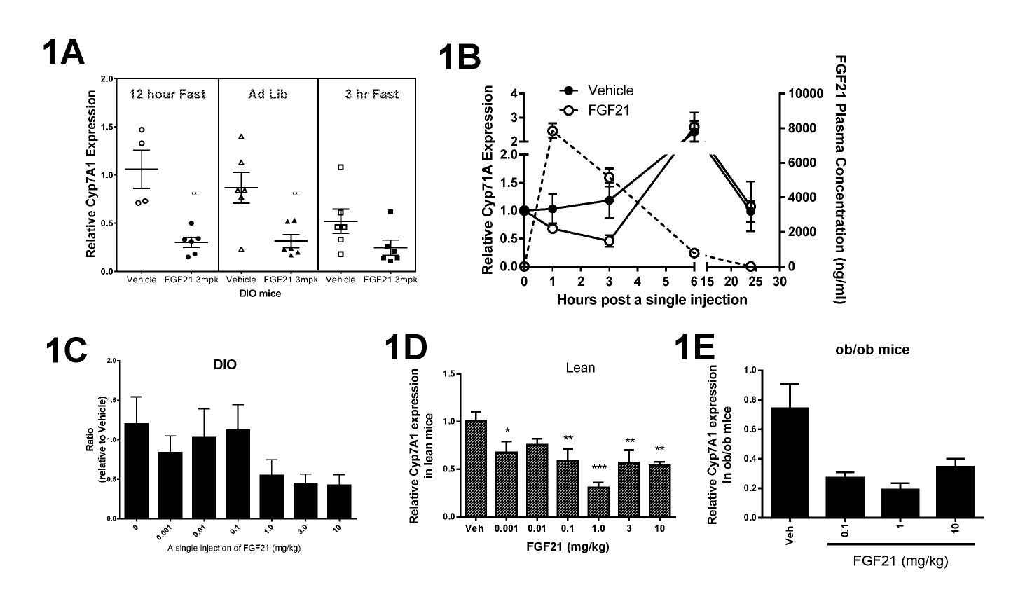

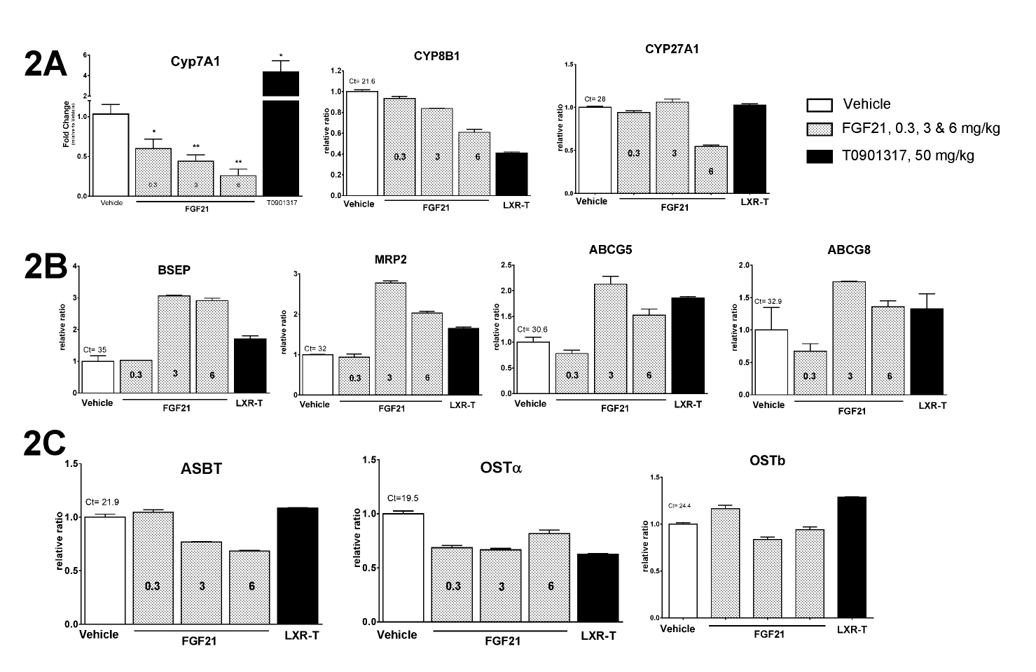

In FIG. 1A, this study examined the effect of FGF21 on CYP7A1 expression under different feeding conditions in DIO mice fed either ad libitum or fasted for a total of 3 or 12 hours. Mice were administered a single-injection of FGF21 (3 mg/kg, IP) 3 hours prior to termination for each condition. CYP7A1 levels were reduced under all three conditions following FGF21 administration, compared to CYP7A levels in Vehicle treated mice. CYP7A levels were reduced by 72% in 12-hour fasted mice, 64% in ad-lib fed mice, and 52% in 3-hour fasted mice.

In FIG. 1B, CYP7A1 expression was measured over a time-course of 24 hours in DIO mice following a single-injection of FGF21 (3 mg/kg, IP). Plasma and liver samples were collected at 1, 3, 6, and 24 hours post-injection. For each time point, mean CYP7A1 expression levels are plotted against respective mean FGF21 plasma concentrations from the same mice. Following FGF21 administration, CYP7A1 levels were reduced by 34% and 61% at the 1 and 3 hour time points, coinciding with the peak FGF21 serum concentrations. By the 6-hour and 24-hour time points, CYP7A1 expression level was nearly identical between Vehicle- and FGF21-treated groups coinciding with clearance of FGF21 from the serum. FGF21 serum concentration was 10-fold less at the 6-hour time point than at the 1-hour time point and was below quantifiable levels by the 24-hour time point.

The dose-response effect of FGF21 on CYP7A expression was examined in DIO (FIG. IC), lean (FIG. 1D) and ob/ob (FIG. 1E) mice. DIO and lean C57BL6 mice received FGF21 at (0, 0.001, 0.01, 0.1, 1.0, 3.0, and 10 mg/kg. IP). Terminal liver samples were collected 3-hours post-injection from mice fasted for 3-hours. In DIO mice, CYP7A1 expression was reduced by 54% in mice administered with FGF21 at 1.0 mg/kg and maximal CYP7A1 reduction of 65% was achieved in mice administered with FGF21 at 10 mg/kg. In Lean C57BL6 mice, CYP7A1 expression was reduced by 33% in mice administered FGF21 (0.001 mg/kg) and maximal CYP7A1 reduction of 47% was achieved in mice administered FGF21 at (10 mg/kg).

In addition, CYP7A1 expression was measured in ob/ob mice administered with FGF21 at (0, 0.1, 1, and 10 mg/kg. IP). Terminal liver samples were collected 4-hours post-injection from ad lib fed mice. Reduction in CYP7A1 expression ranged from 53% to 74% in mice administered FGF21.

### Acute Effect of Recombinant FGF21 on Multiple Genes Involved in Bile Acid Synthesis, Secretion, and Re-Absorption in DIO Mice

A study was conducted to evaluate the effects of recombinant FGF21 on genes related to bile acid synthesis, excretion, and intestinal absorption in DIO mice. DIO mice were conditioned as described in Example 1 and were stratified into treatment groups based on body weight. Mice were administered a single-injection (IP) of recombinant FGF21 at 0.3, 3 and 6 mg/kg or a single oral gavage of a Liver X Receptor agonist (LXR, T0901317, 50 mg/kg, Cayman Chemical, CAS 293754-55-9). Food was removed and liver, gallbladder, and ileum samples were collected 3-hours post-injection for gene expression analysis. The ileal samples were flushed clean with saline. All tissue samples were snap-frozen in liquid nitrogen.

In FIG. 2A, gene expression analysis was performed on liver samples for genes related to bile acid synthesis. Acute administration of FGF21 dose-dependently inhibited hepatic expression of CYP7A1 and CYP8b1, both key genes in the classic bile acid synthesis pathway. Following FGF21 administration, CYP7A1 expression was reduced by 42% (0.3 mg/kg), 57% (3 mg/kg), and 75% (6 mg/kg). CYP8B1 expression was reduced by 7% (0.3 mg/kg), 16% (3 mg/kg), and 39% (6 mg/kg). CYP27A1, a key gene in the alternative bile acid synthesis pathway, was also suppressed by 45% in mice treated with the highest dose of FGF21 (6 mg/kg). As expected, CYP7A1 expression increased 4-fold in DIO mice treated with LXR agonist (T0901317) compared to DIO mice treated with Vehicle.

In FIG. 2B, gene expression analysis was performed in genes related to bile acid excretion in gallbladder samples. FGF21 administration increased the expression of genes involved in bile acid, phospholipid, and sterol transport in the gall bladder of DIO mice. Mice administered with higher doses of FGF21 (3 and 6 mg/kg, IP) increased the expression of bile acid transporter, BSEP, by 200%, phospholipid transporter, MRP2, by 177%, and sterol transporter. ABCG5 and ABCG8, by 112% and 75%, respectively. These 4 genes were also up-regulated in DIO mice treated with LXR agonist (T0901317, PO) compared to DIO mice treated with Vehicle.

In FIG. 2C, gene expression analysis was performed in genes related to ileal bile acid re-absorption. OST□ gene expression was reduced in DIO mice dosed with either 3 doses of FGF21 (0.3, 3, and 6 mg/kg, IP) or the LXR agonist (T0901317, PO) compared to DIO mice treated with Vehicle. Maximal reduction in OST□ gene expression was 33% in FGF21 treated mice. ASBT gene expression was dose-dependently reduced in FGF21 treated mice with maximal reduction of 32%0/observed in mice treated with FGF21 6 mg/kg. A marginal reduction in OST□ gene expression was observed in DIO mice treated with FGF21 (3 and 6 mg/kg) with a maximal reduction of 16%.

### Effect of Recombinant FGF21 and AMG 876 Surrogate on Bile Acid Levels in Lean C57BL6 Mice

A nine day study was conducted with multiple injections of recombinant FGF21 or recombinant AMG 876 Surrogate to evaluate the effects on bile acid levels in 18 week old lean C57BL6 mice. FGF19 was also included as a comparison. Lean mice were maintained on standard chow diet and were stratified into treatment groups based on body weight. Mice were administered by IP injection twice a day with vehicle, hrFGF19 (0.3 and 3 mg/kg) or hrFGF21 (0.3 and 3 mg/kg). A long-acting FGF21 analog, AMG 876 Surrogate, was IP administered at 1 and 10 mg/kg every 3 days. Mice treated with AMG 876 Surrogate received saline injections (IP) when not dosed with test article to ensure all study mice received the same number of injections. Three-day total feces were collected during the treatment period from day 0-3 and from day 6-9. At the termination on Day 9, mice received the last drug dose and were placed into new cages without food. Terminal tissue samples were collected 3-hours post the morning test article administration. The liver was snap frozen in liquid nitrogen and the gallbladder was ligated and weighed. An incision was made to the gallbladder and bile was collected following centrifugation. The empty gallbladder was again weighed and the difference between the filled and empty gallbladder was recorded as the bile volume. The small intestine and colon were collected with contents intact. Tissues were individually extracted in 75% ethanol in a volume that was 5-8 times the tissue weight depending on the bile acid contents in each tissue. Bile acid measurements were performed using Crystal Chem mouse bile acid kits (Downers Grove, Ill., cat#80370).

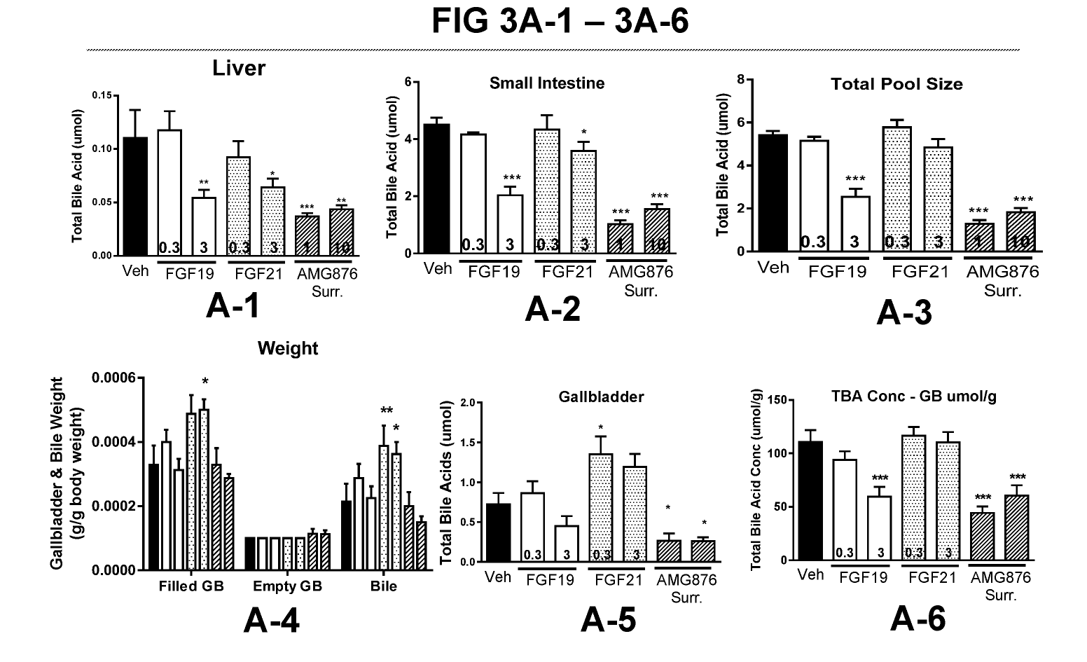

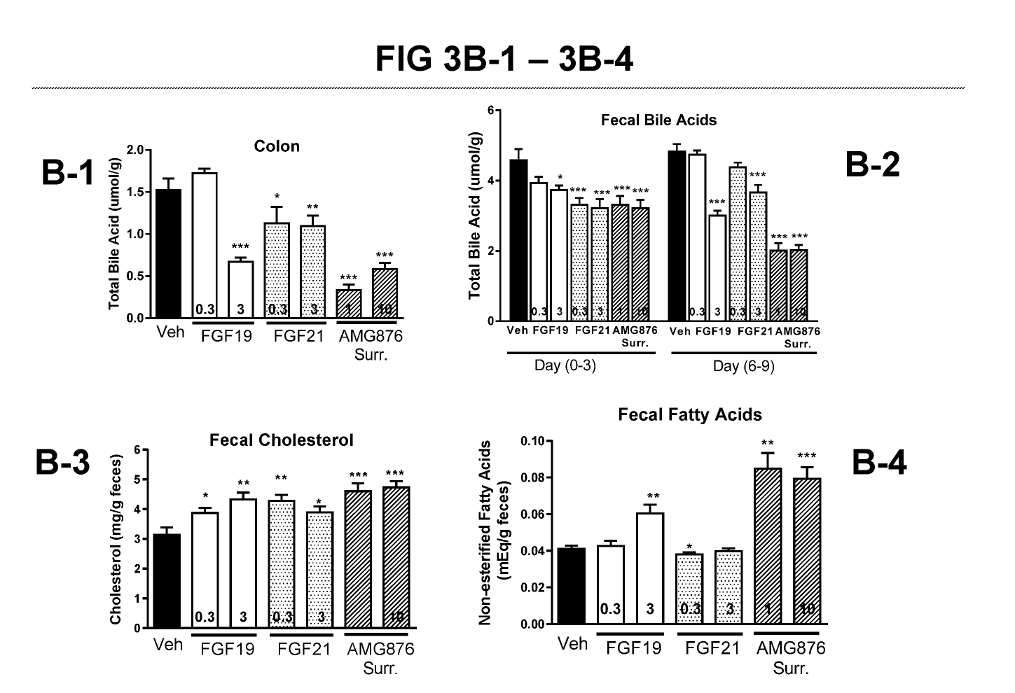

In FIG. 3A, a reduction in total bile acids was observed in the liver and small intestine of mice treated with high dose of FGF19 and FGF21 (3 mg/kg) as well as in mice treated with the low and high doses of AMG 876 Surrogate (1 and 10 mg/kg). A Reductions in bile acid concentrations in the gallbladder and the total bile acid pool size were observed in mice treated with high dose of FGF19 (3 mg/kg) and with the low and high doses of AMG 876 Surrogate. Compared with FGF19, native FGF21 was not as efficacious when administered at the same dose level. However, with the half-life extension and the improvement in potency, the long-acting FGF21 analog, AMG 876 surrogate, achieved the efficacy similar to or slightly better than FGF19. Reductions in total bile acids from liver (67%), small intestine (77%), bile (64%), and total bile acid pool size (76%) were observed in mice treated with AMG 876 Surrogate. The empty gallbladder weight was nearly identical in mice across all treatment groups. However, an increased bile weight, indicative of increased bile volume or bile secretion, was seen in mice treated with both doses of FGF21.

In FIG. 3B, total bile acid concentrations in the colon and feces as well as fecal lipids were measured from terminal colon samples and from fecal samples collected from Day 0-3 and Day 6-9. Total bile acid concentrations in the colon and feces mimicked the profile of total bile acids in the liver, small intestine, and overall pool size (FIG. 3A). Reduction in total bile acid concentrations in the colon and feces was observed in mice treated with FGF19 (3 mg/kg), FGF21 (0.3 and 3 mg/kg) and AMG 876 Surrogate (1 and 10 mg/kg). A maximal reduction of 78% in total bile acid concentration in the colon was observed in mice treated with AMG 876 Surrogate when compared to Vehicle treated mice. Within the first 3 days of treatment initiation, fecal total bile acid concentrations were reduced in mice from all treatment groups with a 30% maximal reduction observed in mice treated with AMG 876 Surrogate compared to the level in mice treated with Vehicle. By day 6-9, fecal total bile acid concentrations in mice treated with FGF19 (0.3 mg/kg) and FGF21 (0.3 and 3 mg/kg) returned to near Vehicle treated levels although FGF21 (3 mg/kg) treated mice were still significantly lower compared to the vehicle group. Further reduction from Day 0-3 to Day 6-9 in fecal total bile acid concentrations were observed in mice treated with FGF19 (3 mg/kg) and AMG 876 Surrogate (1 and 10 mg/kg). Fecal total bile acid concentrations were maximally reduced by 58% in AMG 876 Surrogate treated mice compared to Vehicle. Bile acids are required for lipid solublization and absorption in the intestinal lumen. As would be expected, a reduction in bile acids in the intestinal lumen resulted in a reduced absorption and increased fecal excretion of cholesterol and fatty acids. Fecal cholesterol levels were increased in mice from all treatment groups, including FGF19 and FGF21, and AMG 876. Fecal fatty acid levels were increased in mice treated with high dose FGF19 (3 mg/k) and both low and high dose of AMG 876 Surrogate (1 and 10 mg/kg). Maximal increase in fecal cholesterol (51%) and fecal fatty acids (107%) was observed in mice treated with AMG 876 Surrogate when compared to mice treated with Vehicle.

### Effect of Recombinant AMG 875 and Recombinant AMG 876 on Plasma Bile Acids and C4 Levels in Cynomolgus Monkeys

A chronic dose-escalation study with long-acting FGF21 analogs were conducted in impaired-glucose tolerant cynomolgous monkeys using a dose-escalation protocol. Briefly, animals were individually housed in a controlled environment with 12:12-h light-dark cycle, controlled humidity range of 60% to 80%, and temperature was maintained in the range of 18□C to 26□C. Animals were fed twice a day with a snack in between meals and had free-access to drinking water. Animals were acclimated to all experimental procedures prior to study initiation. Vehicle, AMG 875, or AMG 876 was administered weekly by subcutaneous injection for 9 consecutive weeks. The dose was escalated every 3 weeks (0.3 mg/kg for the first 3 weeks, followed by 1 mg/kg for the next 3 weeks, and 3 mg/kg for the last 3 weeks). Blood samples from cynomolgus monkeys fasted overnight were collected at pre-dose day 14, and on days 5, 12, 19, 26, 33, 40, 47, 54, and 61 (at approximately 117 hours after each weekly dose). During the drug-washout phase of the study, blood samples were collected on days 70, 77, 84, 91, 98, 105 and 133. All fasting samples were subsequently analyzed for plasma total bile acids. In addition, fasting samples from pre-dose day −14, and days 19, 40, and 61 were used to measure 7α-Hydroxy-4-Cholesten-3-One (C4) levels, a biomarker for bile acid synthesis, by LC-MS/MS.

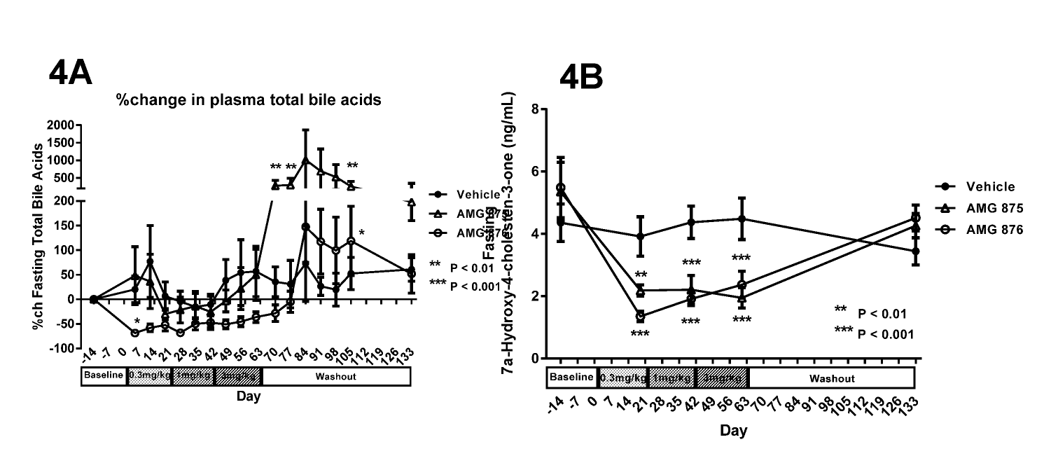

In FIG. 4, plasma total bile acid levels were measured from weekly plasma collections including the 10-week washout period and plotted as the percent change from baseline values. Monkeys treated with AMG 876 demonstrated reduced total bile acid levels across the entire 9-weeks of dosing with a maximal reduction of 69%. Monkeys treated with AMG 875 trended to be lower than monkeys treated with Vehicle. Following 1-week of drug washout, total bile acid levels in monkeys treated with AMG 875 rebounded sharply nearly 5-7 folds over baseline levels. AMG 876, with a superior pharmacokinetic profile over AMG 875, took 3-weeks of drug washout for total bile acid levels to return to levels seen in Vehicle treated monkeys. C4 levels were also measured from fasting plasma samples collected prior to dosing (Day −14) and following the third injection of each dose level at study days (19, 40, 61, and 133). Monkeys treated with both AMG 875 and AMG 876 demonstrated significant inhibition of C4 at each dose level with maximal reductions observed in AMG 875 (57%) and AMG 876 (65%) compared to monkeys treated with Vehicle. C4 levels returned to levels seen in monkeys treated with vehicle by 10 weeks of drug washout.

While the present invention has been described in terms of various embodiments, it is understood that variations and modifications will occur to those skilled in the art. Therefore, it is intended that the appended claims cover all such equivalent variations that come within the scope of the invention as claimed. In addition, the section headings used herein are for organizational purposes only and are not to be construed as limiting the subject matter described.

All references cited in this application are expressly incorporated by reference herein.

What is claimed is:

1. A method of treating a patient with excess bile acid with an extended half-life agonist of the FGF21 signaling pathway.

2. The method of claim 1, wherein the agonist is an FGF21 fusion protein comprising an Fc a linker and FGF21.

3. The method of claim 2, wherein the FGF21 further comprises a point mutation in position of SEQ ID NO: 1 at lysine 98 to arginine and proline 171 to glycine.

4. The method of claim 3, wherein the FGF21 further comprises a point mutation at arginine 180 to glutamic acid.

5. The method of any of claims 1-4, wherein the agonist has a half-life of greater than 5 hours.

6. The method of any of claims 1-5, wherein upon administration of the agonist, bile acid is reduced by a statistically significant amount relative to pre-treatment levels.

7. The method of claim 6, wherein upon administration of the agonist, the CYP7A1 biomarker of bile acid production is reduced by a statistically significant amount relative to pre-treatment levels.

8. The method of any of claims 1-7, wherein the condition to be treated is selected from progressive familial intrahepatic cholestasis type 2 and 3 (BSEP and MDR3 mutations respectively; these are pumps that export bile acids and phospholipid out of liver), intrahepatic cholestasis of pregnancy (ICP), drug-induced cholestasis, contraceptive-induced cholestasis, primary biliary cirrhosis (autoimmune), primary sclerosing cholangitis (autoimmune), cryptogenic biliary fibrosis/cirrhosis, total parenteral nutrition (TPN)-induced cholestasis, bile duct injury following liver transplantation, sepsis-associated cholestasis, progressive sclerosing cholangitis, idiopathic adulthood ductopenia, oriental cholangiohepatitis, and cholangiopathy associated with primary hepatolithiasis.

9. An extended half-life agonist of the FGF21 signaling pathway for use in treating a patient with excess bile acid.

10. The use of claim 9, wherein the agonist is an FGF21 fusion protein comprising an Fc a linker and FGF21.

11. The use of claim 10, wherein the FGF21 further comprises a point mutation in position of SEQ ID NO: 1 at lysine 98 to arginine and proline 171 to glycine.

12. The use of claim 11, wherein the FGF21 further comprises a point mutation at arginine 180 to glutamic acid.

13. The use of any of claims 9-12, wherein the agonist has a half-life of greater than 5 hours.

14. The use of any of claims 9-13, wherein upon administration of the agonist, bile acid is reduced by a statistically significant amount relative to pre-treatment levels.

15. The use of claim 14, wherein upon administration of the agonist, the CYP7A1 biomarker of bile acid production is reduced by a statistically significant amount relative to pre-treatment levels.

16. The use of any of claims 9-15, wherein the condition to be treated is selected from progressive familial intrahepatic cholestasis type 2 and 3 (BSEP and MDR3 mutations respectively; these are pumps that export bile acids and phospholipid out of liver), intrahepatic cholestasis of pregnancy (ICP), drug-induced cholestasis, contraceptive-induced cholestasis, primary biliary cirrhosis (autoimmune), primary sclerosing cholangitis (autoimmune), cryptogenic biliary fibrosis/cirrhosis, total parenteral nutrition (TPN)-induced cholestasis, bile duct injury following liver transplantation, sepsis-associated cholestasis, progressive sclerosing cholangitis, idiopathic adulthood ductopenia, oriental cholangiohepatitis, and cholangiopathy associated with primary hepatolithiasis.


SEQUENCE LISTING (15 sequences)

SEQ ID NO: 1 - protein, 181 aa, Homo sapiens
  mat_peptide (1)..(181)
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPAPPEPPGILAPQPPDVGSSDPLSMVGPSQGRSPSYAS

SEQ ID NO: 2 - DNA, 546 bp, Homo sapiens
  misc_feature: Human FGF21 DNA
caccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 3 - protein, 424 aa, ARTIFICIAL SEQUENCE
  AMG 876 Protein
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

SEQ ID NO: 4 - protein, 424 aa, ARTIFICIAL SEQUENCE
  AMG 875 Protein
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYAS

SEQ ID NO: 5 - protein, 15 aa, ARTIFICIAL SEQUENCE
  Protein Linker
GGGGGSGGGSGGGGS

SEQ ID NO: 6 - protein, 15 aa, ARTIFICIAL SEQUENCE
  Protein Linker
GGGGSGGGGSGGGGS

SEQ ID NO: 7 - protein, 8 aa, ARTIFICIAL SEQUENCE
  Protein Linker
GGGNGSGG

SEQ ID NO: 8 - protein, 8 aa, ARTIFICIAL SEQUENCE
  Protein Linker
GGGNGSGG

SEQ ID NO: 9 - protein, 8 aa, ARTIFICIAL SEQUENCE
  Protein Linker
GGGCGGGG

SEQ ID NO: 10 - protein, 5 aa, ARTIFICIAL SEQUENCE
  Protein Linker
GPNGG

SEQ ID NO: 11 - protein, 228 aa, ARTIFICIAL SEQUENCE
  IgG Fc
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 208 aa, Homo sapiens
  MISC_FEATURE: Full Length Human FGF21
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 13 - DNA, 627 bp, Homo sapiens
  misc_feature: Full Length Human FGF21 DNA
atggactcggacgagaccgggttcgagcactcaggactgtgggtttctgtgctggctggtctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 14 - DNA, 40 bp, ARTIFICIAL SEQUENCE
  PCR Primer for Preparing FGF21 Construct
aggaggaataacatatgcatccaattccagattcttctcc

SEQ ID NO: 15 - DNA, 33 bp, Homo sapiens
  misc_feature: PCR Primer for Preparing FGF21 Construct
tagtgagctcgaattcttaggaagcgtagctgg